(19) United States
(12) Patent Application Publication
(10) Pub. No.: US 2021/0094954 A1
(43) Pub. Date: Apr. 1, 2021

(54) PREPARATION METHODS AND APPLICATIONS OF 3,5-DISUBSTITUTED METHYLPYRAZOLO[1,5-A]PYRIMIDINE-7-HYDROXYLIC SALTS

(71) Applicant: Lifeng Xu, San Diego, CA (US)

(72) Inventor: Lifeng Xu, San Diego, CA (US)

(21) Appl. No.: 16/589,141

(22) Filed: Oct. 1, 2019

(51) Int. Cl. C07D 487/04; A61K 31/519; A61K 31/496

(52) U.S. Cl. CPC C07D 487/04; A61K 31/496; A61K 31/519

(57) ABSTRACT

The invention provides a compound of the general formula (1), a derivative or the analog and a preparation method thereof. The compound is synthesized by the reaction of 3-(2,4-difluorophenyl)-5-substituted-2-methylpyrazolo[1,5-α] pyrimidine-7-hydroxy with a base or an alkali metal and the compound has antibacterial and antifungal activities. Therefore this invention also provides the use thereof as an antibacterial and antifungal disease drug.

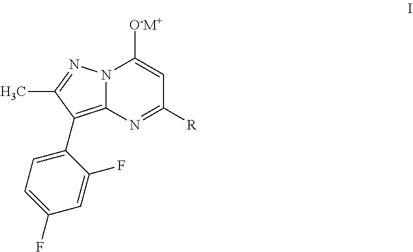

### FIELD OF THE INVENTION

This invention relates to 3-(2,4-difluorophenyl)-5-substituted-2-methylpyrazolo[1,5-α] pyrimidin-7-hydroxylic salts, derivatives or analogs to their medication applications as antibacterial and antifungal agents, to pharmaceutical compositions containing these compounds and to methods for their preparation.

### BACKGROUND OF THE INVENTION

Due to the abuse of a large of antibacterial drugs, the effective antibacterial drugs may have drug-resistant strains, which are resistant to various antibacterial drugs, such as methicillin-resistant Staphylococcus aureus (called super bacteria. MRSA), vancomycin-resistant enterococci (VRE), etc. The spread of infection “super bacteria” has become a serious threat to human health therefore the development of new active antibacterial drugs against resistant bacteria is already imminent.

By search of all the literatures so far there is no any found literature of antibacterial and antifungal properties of 3-(2,4-difluorophenyl)-5-substituted-2-methylpyrazolo[1,5-α]pyrimidin-7-hydroxylic salts except the similarity of parent structure with the total different pharmacological activities. Anti-inflammation activity was described by WO 2008062026, EP 2086947 and US 20130252951. The treatment of liver dysfunction was described by US20160022742. The treatment of liver dysfunction was described by US20160022742. All the above literature patens are irrelevant to the present inventions either their biological activity or their compounds' structures.

In summary, all the literatures have been searched to date, in terms of chemical structure, synthesis and therapeutic applications, there is no any report or patent of the antibacterial and antifungal bioactive applications with present invention of 3-(2,4-difluorophenyl)-5-substituted-2-methylpyrazolo[1,5-α]pyrimidin-7-hydroxylic salts, derivatives or analogs thereof represented by the general formula (I) to their medication applications as antibacterial and antifungal agents, to pharmaceutical compositions containing these compounds and to methods for their preparation.

### DETAILED DESCRIPTION OF THE INVENTION

The purpose of the present invention is to provide a 3-(2,4-difluorophenyl)-5-substituted-2-methylpyrazolo[1,5-α]pyrimidin-7-hydroxyl salt compound, derivatives and analogs to their use as antibacterial and antifungal agents, to pharmaceutical compositions containing these compounds and to methods for their preparation, which have the following general formula I

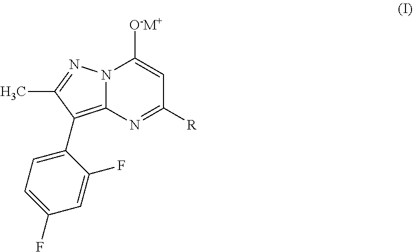

wherein M⁺ is one of Li⁺, K⁺, Na⁺, Ca^{+/2} and R is H, optionally substituted aromatic, alicyclic or fused romatic and alicyclic groups.

In one embodiment, R in compound (I) is one of the following groups 1, 2, 3, 4, 5, 6, 7, 8, and 9:

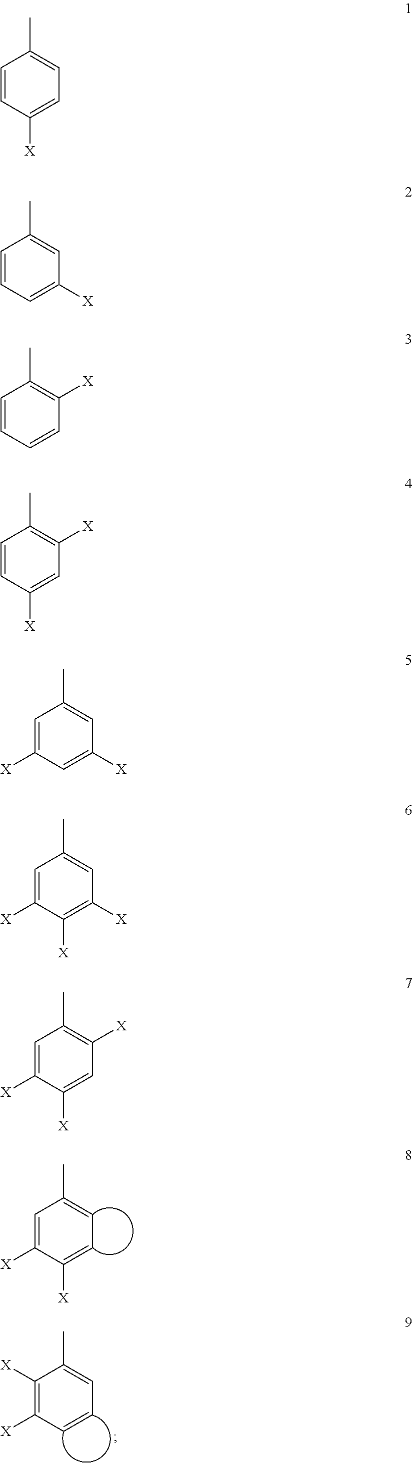

wherein the alicyclic ring fused with the aromatic ring in the chemical structure of 8 and 9 is a three-carbon, four-carbon or five-carbon alicyclic ring; X may not exist, or may be the same or different substituents: optionally substituted hydrogen, halogen, hydroxyl, mercapto, cyano, carbonyl, substituted carbonyl, aldehyde, ketone, nitro, carboxyl, substituted carboxyl, carboxylic acid ester, secondary amino, tertiary amino, two-carbon, three-carbon, four-carbon or five-carbon or six-carbon alkoxy, arylalkoxy, aryloxy, heteroaryloxy, alkylthio, mercaptoester, arylalkylthio, arylthio, heteroarylthio, ester, acyloxy, phosphoxy, sulfoxy, aryloxy, quaternary ammonium salt, amide, hydrazino, oxime, hydrazone, nitrogen-containing aliphatic, nitrogen-containing aromatic, nitrogen-containing cyclic, nitrogen-containing alicyclic, nitrogen-containing aromatic cyclic, nitrogen-containing aromatic heterocyclic, phosphide, phosphoric acid, phosphoric acid ester, one-carbon, two-carbon, three-carbon, four-carbon or five-carbon group or a combination thereof.

In a second aspect, the present invention discloses a process for the preparation of the above compound (I), comprising:

i) For the preparation of the key intermediates (3) by the cyclization of the compound (1) with the compound (2) containing the R group at a temperature of −40 to 180° C. in an organic solvent under the catalysis

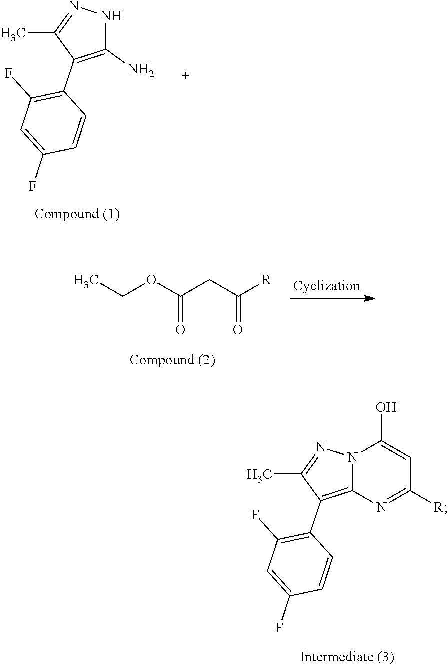

ii) For the preparation of the compound (I) by the reaction of the key intermediates (3) with an alkali or an alkali metal at a temperature of −40 to 180° C. in an organic solvent under the catalysis.

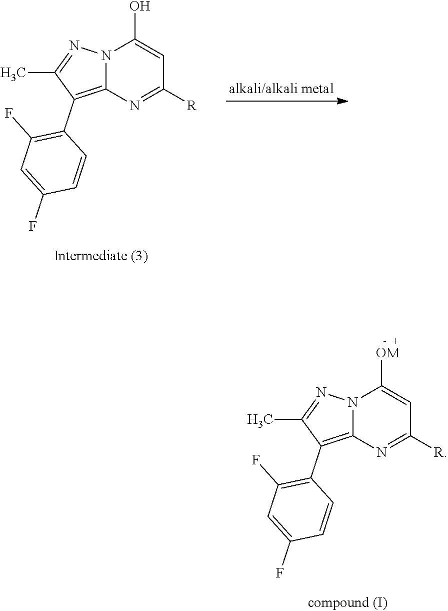

In one embodiment, Step i) the catalyst is an acid or a base catalyst and the acid catalyst is selected from the group consisting of hydrochloric acid, sulfuric acid, nitric acid, fumaric acid, maleic acid, succinic acid, acetic acid, citric acid, tartaric acid, carbonic acid, phosphoric acid and oxalic acid; the base is selected from the group consisting of sodium carbonate, sodium hydride, sodium hydroxide, potassium hydroxide, triethylamine, pyridine, piperidine and N-methylpyrazine;

In one embodiment, Step ii) the solvent is selected from one of tetrahydrofuran, 1,4-dioxane, acetonitrile, N,N-dimethylformamide, N,N-dimethylacetamide, n-hexane and toluene, Preferably N,N-dimethylformamide. the base is selected from the group consisting of sodium carbonate, sodium hydride, potassium hydroxide, sodium hydroxide or calcium hydroxide, and the alkali metal is selected from the group consisting of Li, Na or K.

In a third aspect, the present invention discloses a method of treating, preventing or slowing the progression of bacteria, fungi and other infection diseases associated with a bacterial or fungal infection, comprising: a therapeutically effective amount of the compound (I), a derivative or an analog according to the claim 1 thereof is administered to a patient in need of such treatment.

In one embodiment, the bacterium may be a Gram-positive bacteria: Staphylococcus, pneumococci, Enterococcus faecalis, Streptococcus, Streptococcus bovis, Streptococcus pneumoniae, Streptococcus pneumoniae, Streptococcus pneumoniae, Streptococcus pneumoniae, Streptococcus pyogenes, Streptococcus agalactiae, Streptococcus mutans, Streptococcus bovis, Streptococcus agalactia B, Streptomyces, diphtheria, tetanus, Escherichia, Bacillus anthracis, tetanus, Bacillus cereus, Bacillus subtilis, Clostridium, Bacillus cereus, Bacillus subtilis, Bacillus anthracis, diphtheria, Clostridium, tetanus, Clostridium perfringens, Clostridium perfringens, atinomycetes, tuberculosis.

Further, the above-mentioned bacteria are Gram-positive resistant bacteria: methicillin-resistant Staphylococcus, vancomycin-resistant Staphylococcus aureus, Staphylococcus-induced clindamycin resistance, vancomycin enterococcus, enterococci high level of aminoglycoside resistance, penicillin-resistant Streptococcus pneumoniae, multi-drug resistant Acinetobacter baumannii, drug resistance and multi-drug resistant Mycobacterium tuberculosis, Mycobacterium tuberculosis, Streptococcus, Enterococcus faecalis, Pseudomonas aeruginosa, Escherichia coli, Acinetobacter baumannii, Haemophilus influenzae-resistant haemophilus, Neisseria gonorrhoeae, Neisseria meningitidis, Enterobacteriaceae-resistant bacteria, drug-resistant patina monocytogenes.

In one embodiment, the disease may be an inflammatory and inflammatory disease associated with a bacterial infection, a fungal and fungal disease, and a complication of viral and viral diseases, immune system diseases due to bacterial and fungal infections.

Further, the diseases is selected from: infections of upper and lower respiratory tract, infections of skin and soft tissue, urinary tract infections, sepsis, endocarditis caused by methicillin-sensitive Staphylococcus, hemolytic Streptococcus and Streptococcus pneumoniae; Haemophilus influenzae, Proteus mirabilis, large intestine urinary tract infections caused by bacterium-sensitive strains and pneumonia; respiratory infections, urinary tract infections, skin soft tissues caused by Streptococcus, Streptococcus pneumoniae, and susceptible strains of Haemophilus influenzae, Escherichia coli, and Proteus mirabilis infection, sepsis, bone and joint infections and abdominal and pelvic infections; infection of hemolytic Streptococcus, pneumococci, sensitive Staphylococcus aureus; endocarditis caused by Streptococcus viridans and enterococcus; and gangrene, anaerobic bacterial infection, anthrax, syphilis, gonorrhea.

In one embodiment, the method of treating, preventing or slowing the progression of diseases with a compound (I), a derivative or an analog is administered together with at least one known drug selected from:

β-lactams: penicillin, procaine penicillin, benzathine penicillin, methicillin, oxacillin, cloxacillin, dicloxacillin sodium, ampicillin, amoxicillin, hetacillin, carbenicillin, sulbenillin, temocillin, furazocillin, piperacillin, azlocillin, mezlocillin, ticarcillin, mecillin, apacillin, apocillin, lenampicillin, flucloxacillin, sulbaccillin, piramacillin, acesulfamecil, bacilcillin, carocycline, furbuterazine, ceftriaxone, cefpirome, cefuroxime, cefuroxime axetil, cefotaxime, cefotaxime, cefotaxime, cephalosporin thiopurine, cefacid, cefpirin, cefazolin, cefmenoxime, cefoperazone, cefaclor, ceftizoxime, ceftazidime, cefonicid, cefdinir, cefixime, cefradine, cefpiamine, cefmenoxime, ceftiram, cefpodoxime, cefdiprozil, cefotiam, cefetamet, ceftizox, cefprozil, ceftibuten, cefepime, cephalexin, cephradine, cefaclor, ceftriaxone, cephalosporin, ampicillin, cefmenudene, cefsulodin, cefoxitin, cefmetazole, cefotetan, cefminox, cephalosporin, cefaclor, cefazolin, cefotiam, ceftriax, cefixime, cefotaxime, chlorocarbon cephalosporin, fluoxetine; macrolides: dirithromycin, roxithromycin, ropoxmycin, clarithromycin, fluoroerythromycin, azithromycin, rotamycin, tacomus, erythromycin, erythromycin, clarithromycin, guitarmycin, melamycin, columnar leucomycin, medimycin, azithromycin, josamycin, spiramycin, acetylspira aminoglycosides: netilmicin, astemizine, arbekacin, isepamicin streptomycin, caramycin, gentamicin, tobramycin, amikacin, cisplatin michelin, neomycin, paromomycin, formimicin, small nomitellin, isepamicin, dibemimethine, dardimethine, spectinomycin, streptomycin, kanamycin, etimicin, dibekacin; amide alcohols: chloramphenicol, amber chloramphenicol, palm chloramphenicol, thiamphenicol, lincomycin, clindamycin, gram polyphosphates; polypeptides: cyclosporine, teicoplanin, polymycin, polymyxin, vancomycin, norvancomycin, bacitracin, polymyxin b, fusidic acid, micammycin; rifamycin: rifabutin, rifapentine, rifaximin, rifampicin, rifamycin, rifampicin; quinolones: enoxacin, toloxacin, norfloxacin, ciprofloxacin, lomefloxacin, serfloxacin, pefloxacin, fleroxacin, temafloxacin, haloxacin, moxifloxacin, valvafloxacin, gepafloxacin, ofloxacin, levofloxacin, pazufloxacin, rufloxacin, sulfisoxazole, sulfamethoxazole, sulfadiazine, sodium sulfacetamide, silver sulfadiazine, trimethoprim, pipemidic acid, nitrofurantoin, furazolidone, nalidixic acid, ofloxacin, gatifloxacin, pazufloxacin, troxafloxacin, moxifloxacin; tetracyclines: tetracycline, terpene, minocycline, chlortetracycline, doxycycline, oxytetracycline, doxycycline, metacycline, dimecycline, indomethacin, beta-lactamase inhibitor clavulanic acid, sulbactam, tazobactam; carbapenemantibiotics: imipenem, cilastatin, panipenem, betamipron, meropenem, cefomycin; sulfonamides: sulfametholone, sulfamethazine, sulfamethazine, sulfamethoxazole, sulfamonomethoxine, iodine-p-methoxypyrimidine, iodine, sulfaguanidine, zinc sulfadiazine, sulfamethamine, amber sulfonamide thiazole, thiazolidine, sulfamethoxazole, thienamycin, clavulanic acid, aztreonam, imipenem, faropenem, cilastatin, sulbactam, tazobactam, carumimo south, sirmimycin, chloramphenicol palmitate, fosfomycin, SV, bromoprolin, octenidine, urotropine, montadylamine, bismuth subsalicylate, metronidazole phosphate disodium, sulpirone, new sterilization, metronidazole, arubicin, epirubicin, zorubicin, pirarubicin, idarubicin, mupirocin, nimidazole, tinidazole, pipemidic acid, nitrofurantoin; nitrofuran: furazolidone, trimethoprim; methylfuran: sulfasalazine; antifungal: thiconazole, lanoconazole, noconazole, butoconazole, chlorconazole, fenteconazole nitrate, sheraconazole, oxyxazole, bifonazole, fluconazole, itraconazole, saconazole, clotrimazole, econazole, tioconazole, miconazole, ketoconazole, naftifine, butenafine, ciclopirox, amorolfine, amphotericin B, erythromycin, flucyto sine, terbina fen, nystatin, griseofulvin, and flunin.lactams:penicillin, procaine penicillin, benzathine penicillin, methicillin, oxacillin, cloxacillin, dicloxacillin, hetacillin, sulbenicillin, temocillin, mecillinam, piperacillin, ticarcillin, ticarcillin, flucloxacillin, sultamicillin, phenoxymethylpenicillin, bacampicillin, ticarcillin, talampicillin, furbenicillin, aspoxicillim, pivam-picilli, meticillin, nafcillin, pivmecillinam, lenampicillin varacillin, apalcillin, carindacillin, carbenicillin, ceftriaxone, cefpirome, cefuroxime, ceftazidime, cefotaxime, cephalothin, cefathiamidine, cephalosporin, cephalosporin, cefazolin, cefmenoxime, cefoperazone, cefaclor, ceftizoxime, cefdinir, cefixime, cephradine, cefpiramide, cephalosporin, cephalosporin, cefpodoxime, cefodizime, cefotiam, cefetamet, cefprozil, cephalosporin, cefepime, cephalexin, cephradine, cefadroxil, cefoxitin, cefmeta-zole, cefotetan, cefminox, cephalosporin, cefozopran, cephalosporin, cefalotin, cefaloglycine, cefalexin, cefradine, cefacetrile cefapirin, cefadril, cefroxadine, ceftezole, cefonicid, cefamandole, cefbuperazone, cefdinir, cefzon, cefcapene, cefotaxime, cefteram, cefsulodine, latamoxef, cefpimizole, cefuzonam, aztreonam, erythromycin, roxithromycin, adriamycin, clarithromycin, fluoerythromycin, azithromycin, kitasamycin, albomycin, leucomycin, josamycin, spiramycin, acetyl spiramycin, netilmicin, isepamicin, streptomycin, gentamicin, tobramycin, amikacin, sisomicin, neomycin, neomycin, spectinomycin, streptomycin, kanamycin, chloramphenicol, thiamphenicol, lincomycin, clindamycin, clindamycin, cyclosporine, teicoplanin, vancomycin, teicoplanin, bacitracin, polymyxin B, rifamycin, rifabutin, rifapentine, rifaximin, rifampicin, rifamycinn, enoxacin, norfloxacin, ciprofloxacin, lomefloxacin, sparfloxacin, pefloxacin, fleroxacin, moxifloxacin, ofloxacin, levofloxacin, rufloxacin, isoxazole, sulfamethoxazole, sulfadiazine, trimethoprim, pipemidic acid, nitrofurantoin, furazolidone, nalidixic acid, difloxacin, gatifloxacin, pazufloxacin, moxifloxacin acid, tetracycline, minocycline, chlortetracycline, doxycycline, oxytetracycline, doxycycline, metacycline, clavulanic acid, sulbactam, tazobactam, imipenem, cilastatin, panipenem, betamipron, meropenem, cephamycin, sulfamethoxazole, sulfamethazine, sulfonamides, sulfamonomethoxine, sulfamethoxazole, sulfathiazole, sulfamethoxazole, sulfathiazole, thienamycin, aztreonam, faropenem, cilastatin, tazobactam, streptomycin, neomycin, kanamycin, neomycin, clindamycin, fosfomycin, brodimoprim, metronidazole, aclarubicin, epirubicin, pirarubicin, mupirocin, tinidazole, sulfasalazine, itraconazole, bifonazole, fluconazole, clotrimazole, econazole, miconazole, ketoconazole, naftifine, butenafine, ciclopirox, amorolfine, amphotericin b, flucytosine, terbinafine, nystatin, gentamycin, nebramycin, micronomicin, ribostamycin, astromicin, dibekacin, etimicin, nofloxacin, cotrim, sulfamethoxazole, mafenide, brodimoprim clafalix, laurylin, azitromycin, midecamycin, acetylmidecamycin, rokitamycin, meleumycin, methacycline, doxycyclme, demeclocycline, nifuratel, methylmercadone, norvancomycim, colistin, gramicidin, isoniazid, ethambutol, pyrazinamide, rifamycin, rifandin, viomycin, rifampin, capreomy-cin, ethionamide, terconazole, fenticonazole, sulconazole, fluorocytosine, ciclopirox, mepartricin, exalamide, terbinafine, ribavirin, acyclovir, ganciclovir, indinavir, nelfinavir, ritonavir, cidofovir, penciclovir, buciclovir, penciclovir, famciclovir, valaciclovir, famotine, vidarabine, zidovudine, azidothymidine, foscarnet, delavirdine, moroxydine, idoxuridine, amantadine, interferons, rimantadine, clindamycim.

In one embodiment, the method of treating, preventing or slowing the progression of diseases with a compound (I), a derivative or an analog is administered at a dose of from 0.02 mg/kg to 250 mg/kg. It can be administered in a variety of ways, for example, by oral, parenteral, subcutaneous, intravenous, intramuscular, intraperitoneal, transdermal, buccal Intrathecal, intracranial, intranasal or topical administration.

### DETAILED DESCRIPTION

The invention will now be further illustrated by the examples, but the following examples are merely illustrative and not limiting. One or more aspects and embodiments may be incorporated in different embodiments, although not explicitly described. That is, all aspects and embodiments can be combined in any manner.

### Compounds

The present application discloses a compound represented by the formula (I) a derivative or analog thereof:

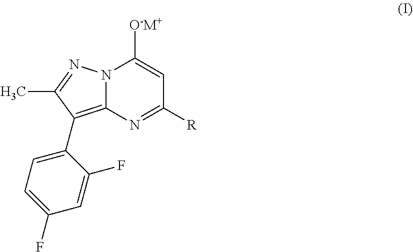

wherein M⁺ is one of Li⁺, K⁺, Na⁺, Ca^{+/2} and R is H, optionally substituted aromatic, alicyclic or fused romatic and alicyclic groups.

Further, R is one of a group 1, 2, 3, 4, 5, 6, 7, 8 and 9;

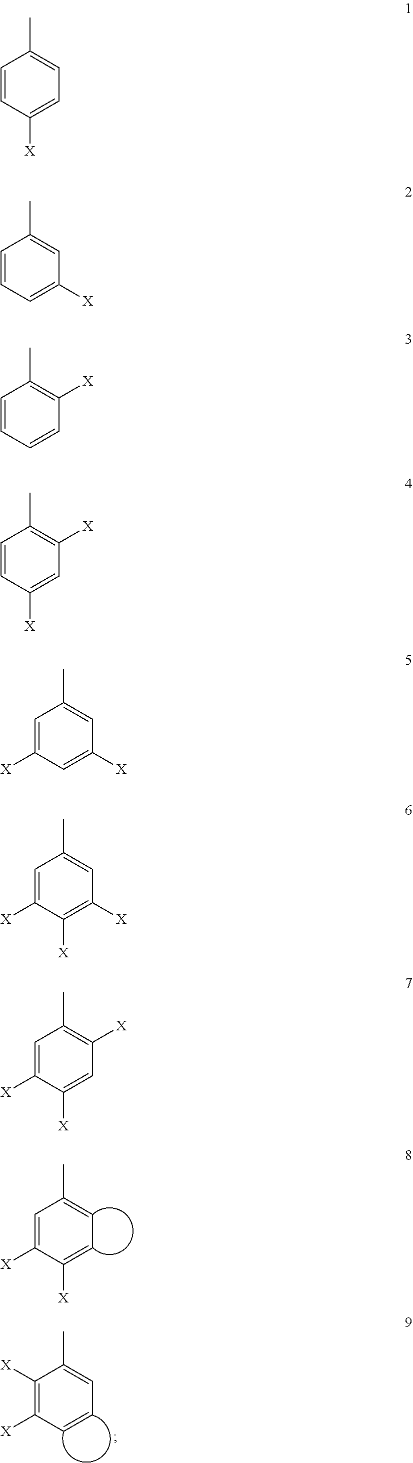

wherein the alicyclic ring fused with the aromatic ring in the chemical structure of 8 and 9 is a three-carbon, four-carbon or five-carbon alicyclic ring; X may not exist, or may be the same or different substituents: optionally substituted hydrogen, halogen, hydroxyl, mercapto, cyano, carbonyl, substituted carbonyl, aldehyde, ketone, nitro, carboxyl, substituted carboxyl, carboxylic acid ester, secondary amino, tertiary amino, two-carbon, three-carbon, four-carbon or five-carbon or six-carbon alkoxy, arylalkoxy, aryloxy, heteroaryloxy, alkylthio, mercaptoester, arylalkylthio, arylthio, heteroarylthio, ester, acyloxy, phosphoxy, sulfoxy, aryloxy, quaternary ammonium salt, amide, hydrazino, oxime, hydrazone, nitrogen-containing aliphatic, nitrogen-containing aromatic, nitrogen-containing cyclic, nitrogen-containing alicyclic, nitrogen-containing aromatic cyclic, nitrogen-containing aromatic heterocyclic, phosphide, phosphoric acid, phosphoric acid ester, one-carbon, two-carbon, three-carbon, four-carbon or five-carbon group or a combination thereof.

One embodiment of the present invention may be any one selected from the group consisting of:

sodium3-(2,4-difluorophenyl)-2-methyl-5-(4-(trifluoromethyl)phenyl)pyrazolo[1,5-α]pyrimidin-7-olate, sodium5-(4-cyanophenyl)-3-(2,4-difluorophenyl)-2-methylpyrazolo[1,5-α]pyrimidin-7-olate, sodium 3-(2,4-difluorophenyl)-2-methyl-5-(4-nitrophenyl)pyrazolo[1,5-α]pyrimidin-7-olate, sodium 3-(2,4-difluorophenyl)-5-(4-methoxyphenyl)-2-methylpyrazolo[1,5-α]pyrimidin-7-olate, sodium3-(2,4-difluorophenyl)-5-(4-(dimethylamino)phenyl)-2-methylpyrazolo[1,5-α]pyrimidin-7-olate, disodium 3-(2,4-difluorophenyl)-2-methylpyrazolo[1,5-α] pyrimidine-5,7-bis(olate), disodium 4-(3-(2,4-difluorophenyl)-2-methyl-7-hydroxy-pyrazolo[1,5-α]pyrimidin-5-yl)benzoate, disodium 4-(3-(2,4-difluorophenyl)-2-methyl-7-oxidopyrazolo[1,5-α]pyrimidin-5-yl) benzenesulfonate; sodium 3-(2,4-difluorophenyl)-2-methyl-5-(4-sulfamoylphenyl)pyrazolo[1,5-α] pyrimidin-7-olate, sodium 3-(2,4-difluorophenyl)-2-methyl-5-(4-phenoxyphenyl)pyrazolo[1,5-α] pyrimidin-7-olate, sodium 5-(4-carbamoylphenyl)-3-(2,4-difluorophenyl)-2-methylpyrazolo[1,5-α] pyrimidin-7-olate, sodium 3,5-bis(2,4-difluorophenyl)-2-methylpyrazolo[1,5-α]pyrimidin-7-olate, sodium 5-(4-acetoxyphenyl)-3-(2,4-difluorophenyl)-2-methylpyrazolo[1,5-α]pyrimidin-7-olate, sodium 3-(2,4-difluorophenyl)-2-methyl-5-(6-(trifluoromethyl)pyridin-3-yl)pyrazolo[1,5-α] pyrimidin-7-olate, sodium 5-(6-cyanopyridin-3-yl)-3-(2,4-difluorophenyl)-2-methylpyrazolo[1,5-α]pyrimidin-7-olate, sodium 3-(2,4-difluorophenyl)-2-methyl-5-(6-nitropyridin-3-yl)pyrazolo[1,5-α]pyrimidin-7-olate, sodium 3-(2,4-difluorophenyl)-5-(6-methoxypyridin-3-yl)-2-methylpyrazolo[1,5-α]pyrimidin-7-olate, sodium 3-(2,4-difluorophenyl)-5-(6-(dimethylamino) pyridin-3-yl)-2-methylpyrazolo[1,5-α]pyrimidin-7-olate, disodium 3-(2,4-difluorophenyl)-2-methy 1-5-(6-hydroxypyridin-3-yl)pyrazolo[1,5-α]pyrimidin-7-olate, potassium 5-(4-cyanophenyl)-3-(2,4-difluorophenyl)-2-methylpyrazolo[1,5-α]pyrimidin-7-olate, disodium 5-(3-(2,4-difluorophenyl)-2-methyl-7-hydroxypyrazolo[1,5-α]pyrimidin-5-yl)picolinate, potassium 3-(2,4-difluorophenyl)-2-methyl-5-(4-(trifluoromethyl)phenyl)pyrazolo[1,5-α]pyrimidin-7-olate, potassium 3-(2,4-difluorophenyl)-2-methyl-5-(4-nitrophenyl)pyrazolo[1,5-α]pyrimidin-7-olate, potassium 3-(2,4-difluorophenyl)-5-(4-(dimethylamino)phenyl)-2-methylpyrazolo[1,5-α]pyrimidin-7-olate, potassium 3-(2,4-difluorophenyl)-2-methyl-5-(4-sulfamoylphenyl)pyrazolo[1,5-α]pyrimidin-7-olate, potassium 3-(2,4-difluorophenyl)-2-methyl-5-(6-(trifluoromethyl)pyridine-3-yl)pyrazolo[1,5-α]pyrimidin-7-olate, potassium 5-(6-cyanopyridin-3-yl)-3-(2,4-difluorophenyl)-2-methylpyrazolo[1,5-α]pyrimidin-7-olate, disodium 5-(3-(2,4-difluorophenyl)-2-methyl-7-hydroxypyrazolo[1,5-α]pyrimidin-5-yl)pyridine-2-sulfinate, sodium 3-(2,4-difluorophenyl)-2-methyl-5-(6-sulfamoylpyridin-3-yl)pyrazolo[1,5-α]pyrimidin-7-olate, sodium 3-(2,4-difluorophenyl)-2-methyl-5-(6-phenoxypyridin-3-yl)pyrazolo[1,5-α]pyrimidin-7-olate, sodium 3-(2,4-difluorophenyl)-5-(4-((2-(dimethylamino)ethyl)carbamoyl)phenyl)-2-methylpyrazolo[1,5-α] pyrimidin-7-olate, sodium 5-(6-carbamoylpyridin-3-yl)-3-(2,4-difluorophenyl)-2-methylpyrazolo[1,5-α] pyrimidin-7-olate, sodium 5-(6-acetoxypyridin-3-yl)-3-(2,4-difluorophenyl)-2-methylpyrazolo[1,5-α]pyrimidin-7-olate, calcium 5-(4-Cyanophenyl)-3-(2,4-difluorophenyl)-2-methylpyrazolo[1,5-α]pyrimidine-7-olate, calcium 3-(2,4-Difluorophenyl)-2-methyl-5-(6-(trifluoromethyl) pyridin-3-yl)pyrazolo[1,5-α]pyrimidine-7-olate.

Pharmaceutically acceptable salts of the compounds of this invention are also within the scope of the invention, the acid of which can be converted to a salt by reaction with a base such as sodium carbonate, sodium hydride, potassium hydroxide or the like. The compound of the present invention has a nitrogen atom structure and is basic and can be reacted to form a salt by reacting with an acid such as hydrochloric acid, fumaric acid, maleic acid, succinic acid, acetic acid, citric acid, tartaric acid, carbonic acid, phosphoric acid, oxalic acid or the like.

Prodrugs of the compounds of the invention are also within the scope of the invention. The medicament of the present invention can be modified into a prodrug to increase its water solubility and molecular volume, and to reduce its toxicity.

### Compound Preparation

### (1) Preparation of Intermediates

For the preparation of the key intermediates (3) by the cyclization of the compound (1) with the compound (2) containing the R group at a temperature of −40 to 180° C. in an organic solvent under the catalysis

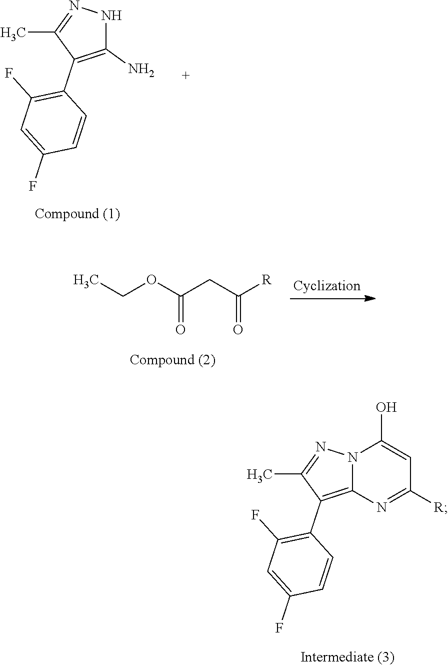

Wherein, the catalyst used may be an acid or a base catalyst; the acid catalyst may be selected from the group consisting of hydrochloric acid, sulfuric acid, nitric acid, fumaric acid, maleic acid, succinic acid, acetic acid, citric acid, tartaric acid, carbonic acid, phosphoric acid and oxalic acid; The base catalyst may be selected from the group consisting of sodium carbonate, sodium hydride, sodium hydroxide, potassium hydroxide, triethylamine, pyridine, piperidine and N-methylpyrazine; the solvent used is tetrahydrofuran, 1,4-dioxane. One of the solvents selected from: acetonitrile, N,N-dimethylformamide, N,N-dimethylacetamide, n-hexane and toluene.

To obtain a key intermediate (3) compound (1) reacts with compound (2) to form a C—N bond, thereby with cyclizion.

(2) The Preparation of 3,5-Disubstituted methylpyrazolo[1,5-α]pyrimidine-7-hydroxylic Salts

The preparation of the compound (I) by the reaction of the key intermediates (3) with an alkali or an alkali metal at a temperature of −40 to 180° C. in an organic solvent under the catalysis.

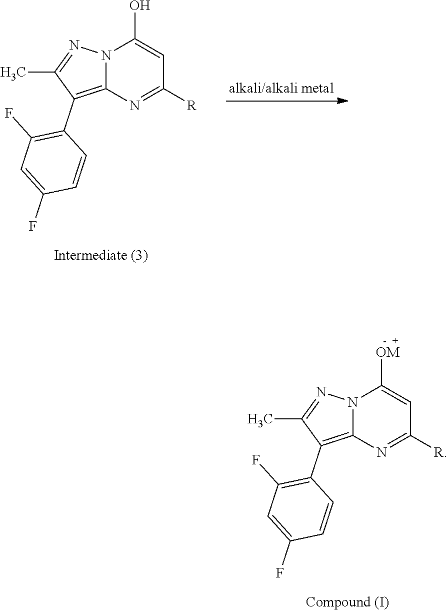

Wherein the solvent used is one of tetrahydrofuran, 1,4-dioxane, acetonitrile, N,N-dimethylformamide, N,N-dimethylacetamide or N,N-dimethylacetamide mixture with water; the base used is sodium carbonate, sodium hydride, potassium hydroxide, sodium hydroxide or calcium hydroxide, and the alkali metal is Li, Na or K.

### Example 1 Preparation of sodium 3-(2,4-difluorophenyl)-2-methyl-5-(4-(trifluoromethyl)-phenyl)pyrazolo[1,5-α]pyrimidin-7-olate

(1) to a mixture of 4-(2,4-difluorophenyl)-3-methyl-1H-pyrazole-5-amine 0.209 g, in N,N-dimethylformamide 20 ml was added ethyl 3-oxo-3-(4-(trifluoromethyl) phenyl)propanoate 0.260 g. The mixture was reacted at 120° C. for 5 h. The reaction solution was filtered and purified by silica gel column chromatography to give the key intermediate, 3-(2,4-difluorophenyl)-2-methyl-5-(4-(trifluoromethyl) phenyl)pyrazolo[1,5-α]pyrimidin-7-ol.

IR (KBr, cm⁻¹) 3246, 2986, 1612, 1565, 1478, 1326, 1171, 1104, 1064, 1014;

¹H NMR (DMSO-d₆) δ 11.53 (b, 1H), 7.75 (d, J=8.0, 1H), 7.72 (d, J=7.5, 2H), 7.68 (d, J=7.5, 2H), 7.07 (d, J=8.0, 1H), 6.82 (s, 1H), 6.74 (s, 1H), 2.06 (s, 3H)

(2) to a mixture of 3-(2,4-difluorophenyl)-2-methyl-5-(4-(trifluoromethyl)phenyl)pyrazolo[1,5-α]pyrimidin-7-ol 0.427 g in mixed solvent of tetrahydrofuran and water (1:1) 20 ml was added sodium hydroxide 0.40 g. The mixture was refluxed for 5 h. The reaction solution was filtered and purified by recrystallization to give the title compound 1.

IR (KBr, cm⁻¹): 3659-2840, 1612, 1565, 1517, 1479, 1419, 1375, 1326, 1263, 1173, 1104, 1063, 1014, 964, 932, 851, 791

¹H NMR (DMSO-d₆) δ7.75 (d, J=8.0, 1H), 7.72 (d, J=7.5, 2H), 7.68 (d, J=7.5, 2H), 7.07 (d, J=8.0, 1H), 6.82 (s, 1H), 6.74 (s, 1H), 2.06 (s, 3H)

### Example 2. Preparation of Sodium 5-(4-cyanophenyl)-3-(2,4-difluorophenyl)-2-methylpyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-(4-cyanophenyl)-3-oxopropanoate 0.217 g catalyzed by dilute hydrochloric acid to give the intermediate 5-(4-cyanophenyl)-3-(2,4-difluorophenyl) 2-methylpyrazolo[1,5-α]pyrimidin-7-ol; the intermediate was further reacted with sodium hydroxide to obtain the title compound 2.

IR (KBr.cm⁻¹): 3661-2840, 2220, 1610, 1566, 1479, 1327, 1175, 1106, 1065, 964

¹H NMR (DMSO-d₆) δ 7.97 (d, J=7.5, 2H), 7.82 (d, J=7.5, 2H), 7.75 (d, J=8.0, 1H), 7.07 (d, J=8.0, 1H), 6.82 (s, 1H), 6.74 (s, 1H), 2.06 (s, 3H)

### Example 3. Preparation of Sodium 3-(2, 4-difluorophenyl)-2-methyl-5-(4-nitrophenyl) pyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-(4-nitrophenyl)-3-oxopropanoate 0.237 g catalyzed by dilute hydrochloric acid to give the intermediate 3-(2,4-difluorophenyl)-2-methyl-5-(4-nitrophenyl) pyrazolo[1,5-α]pyrimidin-7-ol; the intermediate was further reacted with sodium hydroxide to obtain the title compound 3.

IR (KBr.cm⁻¹): 3664-2845, 1614, 1567, 1481, 1329, 1250, 1177, 1106, 1066, 1016

¹H NMR (DMSO-d₆) δ 8.32 (d, J=7.5, 2H), 8.05 (d, J=7.5, 2H), 7.75 (d, J=8.0, 1H), 7.07 (d, J=8.0, 1H), 6.82 (s, 1H), 6.74 (s, 1H), 2.06 (s, 3H)

### Example 4. Preparation of Sodium 3-(2,4-difluorophenyl)-5-(4-methoxyphenyl)-2-methylpyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-(4-methoxyphenyl)-3-oxopropanoate 0.222 g catalyzed by dilute sulfuric acid to give the intermediate 3-(2,4-difluorophenyl)-5-(4-methoxyphenyl)-2-methylpyrazolo[1,5-α]pyrimidin-7-ol; the intermediate was further reacted with sodium hydroxide to obtain the title compound 4.

IR (KBr.cm⁻¹): 3657-2838, 1610, 1563, 1477, 1324, 1250, 1171, 1102, 1061, 962

¹H NMR (DMSO-d₆) δ 7.75 (d, J=8.0, 1H), 7.55 (d, J=7.5, 2H), 7.07 (m, 3H), 6.82 (s, 1H), 6.74 (s, 1H), 3.83 (s, 3H), 2.06 (s, 3H)

### Example 5. Preparation of Sodium 3-(2,4-difluorophenyl)-5-(4-(dimethylamino) phenyl)-2-methylpyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-(4-dimethylamino)-3-oxopropanoate 0.235 g catalyzed by dilute sulfuric acid to give the intermediate 3-(2,4-difluorophenyl)-5-(4-(dimethylamino)phenyl)-2-methylpyrazolo[1,5-α]pyrimidin-7-ol; the intermediate was further reacted with sodium hydroxide to obtain the title compound 5.

IR (KBr.cm⁻¹): 3669-2837, 1610, 1562, 1476, 1340, 1170, 1100, 1060, 1011

¹H NMR (DMSO-d₆) δ 7.75 (d, J=8.0, 1H), 7.61 (d, J=7.5, 2H), 7.07 (d, J=8.0, 1H), 6.85 (d, J=7.5, 2H), 6.82 (s, 1H), 6.74 (s, 1H), 3.06 (s, 6H), 2.06 (s, 3H)

### Example 6. Preparation of Disodium 3-(2,4-difluorophenyl)-2-methylpyrazolo[1,5-α] pyrimidine-5,7-bis(olate)

A procedure similar to Example 1 was employed, but the starting reactant was 4-(2,4-difluorophenyl)-3-methyl-1H-pyrazole-5-amine 0.209 g and ethyl 3-(4-hydroxyphenyl)-3-oxopropanoate 0.208 g catalyzed by dilute sulfuric acid to give the intermediate 3-(2,4-difluorophenyl)-2-methylpyrazolo[1,5-α] pyrimidine-5,7-bis(ol); the intermediate was further reacted with sodium hydroxide to obtain the title compound 6.

IR (KBr.cm⁻¹): 3658-2841, 1612, 1564, 1224, 1172, 1104, 1061, 1012, 960

¹H NMR (DMSO-d₆) δ 7.75 (d, J=8.0, 1H), 7.49 (d, J=7.5, 2H), 7.07 (d, J=8.0, 1H), 6.86 (d, J=7.5, 2H), 6.82 (s, 1H), 6.74 (s, 1H), 2.06 (s, 3H)

### Example 7. Preparation of Disodium 4-(3-(2,4-difluorophenyl)-2-methyl-7-hydroxy-pyrazolo[1,5-α]pyrimidin-5-yl)benzoate

A procedure similar to Example 1 was employed, but the starting reactant was 4-(2,4-difluorophenyl)-3-methyl-1H-pyrazole-5-amine 0.209 g and 4-(3-ethoxy-3-oxopropionyl)benzoic acid 0.236 g catalyzed by dilute fumaric acid to give the intermediate 4-(3-(2,4-difluorophenyl)-2-methyl-7-hydroxypyrazolo[1,5-α]pyrimidin-5-yl)benzoic acid; the intermediate was further reacted with sodium hydroxide to obtain the title compound 7.

IR (KBr.cm−1): 3665-2810, 1617, 1600, 1480, 1326, 1173, 1104, 1063, 1014

¹H NMR (DMSO-d₆) δ 8.10 (d, J=7.5, 2H), 8.0 (d, J=7.5, 2H), 7.75 (d, J=8.0, 1H), 7.07 (d, J=8.0, 1H), 6.82 (s, 1H), 6.74 (s, 1H), 2.06 (s, 3H)

### Example 8. Preparation of Disodium 4-(3-(2,4-difluorophenyl)-2-methyl-7-oxidopyrazolo[1,5-α]pyrimidin-5-yl)benzenesulfonate

A procedure similar to Example 1 was employed, but the starting reactant was 4-(2,4-difluorophenyl)-3-methyl-1H-pyrazole-5-amine 0.209 g and 4-(3-ethoxy-3-oxopropionyl)-benzenesulfonic acid 0.256 g catalyzed by dilute acetic acid to give the intermediate 4-(3-(2,4-difluorophenyl)-2-methyl-7-oxidopyrazolo[1,5-α]pyrimidin-5-yl)benzenesulfonic acid; the intermediate was further reacted with sodium hydroxide to obtain the title compound 8.

IR (KBr.cm−1): 3670-2810, 1612, 1565, 1479, 1326, 1200, 1104, 1063, 1014

¹H NMR (DMSO-d₆) δ 8.01 (d, J=7.5, 2H), 7.75 (d, J=8.0, 1H), 7.64 (d, J=7.5, 2H), 7.07 (d, J=8.0, 1H), 6.82 (s, 1H), 6.74 (s, 1H), 2.06 (s, 3H)

### Example 9. Preparation of Sodium 3-(2,4-difluorophenyl)-2-methyl-5-(4-sulfamoylphenyl)pyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(4-sulfamoylphenyl) propanoate 0.256 g catalyzed by dilute acetic acid to give the intermediate 3-(2,4-difluorophenyl)-2-methyl-5-(4-sulfamoylphenyl)pyrazolo[1,5-α]pyrimidin-7-ol; the intermediate was further reacted with sodium hydroxide to obtain the title compound 9.

IR (KBr.cm−1): 3665-2840, 1612, 1565, 1479, 1320, 1173, 1104, 1063, 1017

¹H NMR (DMSO-d₆) δ 8.07 (d, J=7.5, 2H), 7.92 (d, J=7.5, 2H), 7.75 (d, J=8.0, 1H), 7.07 (d, J=8.0, 1H), 6.82 (s, 1H), 6.74 (s, 1H), 2.06 (s, 3H), 2.0 (b, 2H).

### Example 10. Preparation of Sodium 3-(2,4-difluorophenyl)-2-methyl-5-(4-phenoxyphenyl)pyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(4-(phenoxyphenyl) phenyl)propanoate 0.256 g catalyzed by dilute acetic acid to give the intermediate 3-(2,4-difluorophenyl)-2-methyl-5-(4-phenoxyphenyl)pyrazolo[1,5-α]pyrimidin-7-ol; the intermediate was further reacted with sodium hydroxide to obtain the title compound 10.

IR (KBr.cm−1): 3659-2840, 1612, 1565, 1479, 1326, 1173, 1104, 1063, 1014

¹H NMR (DMSO-d₆) δ 7.60-7.07 (m, 11H), 6.82 (s, 1H), 6.74 (s, 1H), 2.06 (s, 3H)

### Example 11. Preparation of Sodium 5-(4-carbamoylphenyl)-3-(2,4-difluorophenyl)-2-methylpyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-(4-carbamoylphenyl)-3-oxopropanoate 0.235 g catalyzed by dilute phosphoric acid to give the intermediate 5-(4-carbamoylphenyl)-3-(2,4-difluorophenyl)-2-methylpyrazolo[1,5-α]pyrimidin-7-ol; the intermediate was further reacted with sodium hydroxide to obtain the title compound 11.

IR (KBr.cm−1): 3669-2840, 1750, 1565, 1479, 1326, 1173, 1104, 1063, 1014

¹H NMR (DMSO-d₆) δ 8.09 (d, J=7.5, 2H), 7.97 (d, J=7.5, 2H), 7.75 (d, J=8.0, 1H), 7.50 (b, 2H), 7.07 (d, J=8.0, 1H), 6.82 (s, 1H), 6.74 (s, 1H), 2.06 (s, 3H)

### Example 12. Preparation of Sodium 3,5-bis(2,4-difluorophenyl)-2-methylpyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-(2,4-difluorophenyl)-3-oxopro-panoate 0.228 g catalyzed by dilute phosphoric acid to give the intermediate 3,5-bis(2,4-difluorophenyl)-2-methylpyrazolo [1,5-α]pyrimidin-7-ol; the intermediate was further reacted with sodium hydroxide to obtain the title compound 12.

IR (KBr.cm−1): 3669-2850, 1622, 1565, 1481, 1333, 1104, 1063

¹H NMR (DMSO-d₆) δ 7.75 (d, J=7.5, 2H), 7.07 (d, J=7.5, 2H), 6.82 (s, 1H), 6.74 (s, 2H), 2.06 (s, 3H)

### Example 13. Preparation of Sodium 5-(4-acetoxyphenyl)-3-(2,4-difluorophenyl)-2-methylpyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-(4-acetoxyphenyl)-3-oxopro-panoate 0.228 g catalyzed by dilute phosphoric acid to give the intermediate 5-(4-acetoxyphenyl)-3-(2,4-difluorophenyl)-2-methylpyrazolo[1,5-α]pyrimidin-7-ol; the intermediate was further reacted with sodium hydroxide to obtain the title compound 13.

IR (KBr.cm−1): 3652-2838, 1750, 1612, 1565, 1479, 1326, 1173, 1106, 1061, 1014

¹H NMR (DMSO-d₆) δ 7.83 (d, J=7.5, 2H), 7.75 (d, J=8.0, 1H), 7.15 (d, J=7.5, 2H), 7.07 (d, J=8.0, 1H), 6.82 (s, 1H), 6.74 (s, 1H), 2.28 (s, 3H), 2.06 (s, 3H)

### Example 14. Preparation of Sodium 3-(2,4-difluorophenyl)-2-methyl-5-(6-(trifluoromethyl)pyridin-3-yl)pyrazolo[1,5-α] pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(6-(trifluoromethyl) pyridin-3-yl)propanoate 0.261 g catalyzed by dilute sodium hydroxide solution to give the intermediate3-(2,4-difluorophenyl)-2-methyl-5-(6-(trifluoromethyl)pyridin-3-yl)pyrazolo[1,5-α]pyrimidin-7-ol; the intermediate was further reacted with sodium hydroxide to obtain the title compound 14.

IR (KBr.cm−1): 3659-2840, 1612, 1565, 1479, 1326, 1173, 1104, 1063, 1018

¹H NMR (DMSO-d₆) δ 8.58 (s, 1H), 7.88 (d, J=7.5, 1H), 7.75 (d, J=8.0, 1H), 7.35 (d, J=7.5, 1H), 7.07 (d, J=8.0, 1H), 6.82 (s, 1H), 6.74 (s, 1H), 2.06 (s, 3H)

### Example 15. Preparation of sodium 5-(6-cyanopyridin-3-yl)-3-(2,4-difluorophenyl)-2-methylpyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(6-cyanopyridin-3-yl) propanoate 0.218 g catalyzed by dilute sodium hydroxide solution to give the intermediate 5-(6-cyanopyridin-3-yl)-3-(2,4-difluorophenyl)-2-methylpyrazolo[1,5-α]pyrimidin-7-ol; the intermediate was further reacted with sodium hydroxide to obtain the title compound 15.

IR (KBr.cm−1): 3651-2831, 2240, 1612, 1565, 1479, 1326, 1173, 1104, 1063, 1017

¹H NMR (DMSO-d₆) δ 9.20 (s, 1H), 8.35 (d, J=7.5, 1H), 7.75 (d, J=8.0, 1H), 7.73 (d, J=7.5, 1H), 7.07 (d, J=8.0, 1H), 6.82 (s, 1H), 6.74 (s, 1H), 2.06 (s, 3H)

### Example 16. Preparation of sodium 3-(2,4-difluorophenyl)-2-methyl-5-(6-nitropyridin-3-yl)pyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(6-nitropyridin-3-yl) propanoate 0.238 g catalyzed by dilute potassium hydroxide solution to give the intermediate 3-(2,4-difluorophenyl)-2-methyl-5-(6-nitropyridin-3-yl)pyrazolo[1,5-α]pyrimidin-7-ol; the intermediate was further reacted with sodium hydroxide to obtain the title compound 16.

IR (KBr.cm−1): 3651-2840, 1612, 1565, 1479, 1375, 1326, 1173, 1104, 1063, 1017

¹H NMR (DMSO-d₆) δ 9.07 (s, 1H), 8.64 (d, J=7.5, 1H), 8.15 (d, J=7.5, 1H), 7.75 (d, J=8.0, 1H), 7.07 (d, J=8.0, 1H), 6.82 (s, 1H), 6.74 (s, 1H), 2.06 (s, 3H)

### Example 17. Preparation of sodium 3-(2,4-difluorophenyl)-5-(6-methoxypyridin-3-yl)-2-methylpyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(6-methoxypyridin-3-yl)propanoate 0.238 g catalyzed by dilute potassium hydroxide solution to give the intermediate 3-(2,4-difluorophenyl)-5-(6-methoxypyridin-3-yl)-2-methylpyrazolo[1,5-α]pyrimidin-7-ol; the intermediate was further reacted with sodium hydroxide to obtain the title compound 17.

IR (KBr.cm−1): 3649-2840, 1612, 1565, 1479, 1326, 1250, 1173, 1105, 106

¹H NMR (DMSO-d₆) δ7.97 (d, J=7.5, 1H), 7.91 (s, 1H), 7.75 (d, J=8.0, 1H), 7.07 (d, J=8.0, 1H), 6.88 (s, 1H), 6.74 (s, 1H), 6.60 (d, J=7.5, 1H), 3.80 (s, 3H), 2.06 (s, 3H)

### Example 18. Preparation of Sodium 3-(2,4-difluorophenyl)-5-(6-(dimethylamino)pyridin-3-yl)-2-methylpyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(6-(dimethylamino) pyridin-3-yl)propanoate 0.236 g catalyzed by triethylamine dilute solution to give the intermediate 3-(2,4-difluorophenyl)-5-(6-(dimethylamino) pyridin-3-yl)-2-methylpyrazolo[1,5-α] pyrimidin-7-ol; the intermediate was further reacted with sodium hydroxide to obtain the title compound 18.

IR (KBr.cm−1): 3639-2850, 1612, 1565, 1479, 1326, 1200, 1104, 1063, 1021

¹H NMR (DMSO-d₆) δ 8.33 (s, 1H), 7.75 (d, J=8.0, 1H), 7.66 (d, J=7.5, 1H), 7.07 (d, J=8.0, 1H), 6.88 (s, 1H), 6.76 (d, J=7.5, 1H), 6.74 (s, 1H), 3.15 (s, 6H), 2.06 (s, 3H)

### Example 19. Preparation of Disodium 3-(2,4-difluorophenyl)-2-methyl-5-(6-hydroxy pyridin-3-yl)pyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(6-oxidopyridin-3-yl) propanoate 0.209 g catalyzed by triethylamine dilute solution to give the intermediate 3-(2,4-difluorophenyl)-2-methyl-5-(6-hydroxypyridin-3-yl)pyrazolo[1,5-α]pyrimidin-7-ol; the intermediate was further reacted with sodium hydroxide to obtain the title compound 19.

IR (KBr.cm−1): 3659-2840, 1612, 1565, 1479, 1326, 1250, 1173, 1104, 1063, 1014, 964

¹H NMR (DMSO-d₆) δ7.97 (d, J=7.5, 1H), 7.91 (s, 1H), 7.75 (d, J=8.0, 1H), 7.07 (d, J=8.0, 1H), 6.88 (s, 1H), 6.74 (s, 1H), 6.60 (d, J=7.5, 1H), 2.06 (s, 3H)

### Example 20. Preparation of Potassium 5-(4-cyanophenyl)-3-(2,4-difluorophenyl)-2-methylpyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(4-cyanophenyl) propanoate 0.217 g catalyzed by triethylamine dilute solution to give the intermediate 5-(4-cyanophenyl)-3-(2,4-difluorophenyl)-2-methylpyrazolo[1,5-α]pyrimidin-7-ol; the intermediate was further reacted with sodium hydroxide to obtain the title compound 20.

IR (KBr.cm−1): 3661-2840, 2220, 1610, 1566, 1479, 1327, 1175, 1106, 1065, 96

¹H NMR (DMSO-d₆) δ 7.97 (d, J=7.5, 2H), 7.82 (d, J=7.5, 2H), 7.75 (d, J=8.0, 1H), 7.07 (d, J=8.0, 1H), 6.82 (s, 1H), 6.74 (s, 1H), 2.06 (s, 3H)

### Example 21. Preparation of Disodium 5-(3-(2,4-difluorophenyl)-2-methyl-7-hydroxypyrazolo[1,5-α]pyrimidin-5-yl)picolinate

A procedure similar to Example 1 was employed, but the starting reactant was 4-(2,4-difluorophenyl)-3-methyl-1H-pyrazole-5-amine 0.209 g and 5-(3-ethoxy-3-oxopropionyl) pyridinecarboxylic acid 0.237 g catalyzed by triethylamine dilute solution to give the intermediate 5-(3-(2,4-difluorophenyl)-2-methyl-7-hydroxypyrazolo[1,5-α]pyrimidin-5-yl)picolinic acid; the intermediate was further reacted with sodium hydroxide to obtain the title compound 21.

IR (KBr.cm−1): 3659-2840, 1660, 1565, 1479, 1326, 1173, 1104, 1063, 1020

¹H NMR (DMSO-d₆) δ 9.23 (s, 1H), 8.40 (d, J=7.5, 1H), 8.35 (d, J=7.5, 1H), 7.75 (d, J=8.0, 1H), 7.07 (d, J=8.0, 1H), 6.88 (s, 1H), 6.74 (s, 1H), 2.06 (s, 3H)

### Example 22. Preparation of potassium 3-(2,4-difluorophenyl)-2-methyl-5-(4-(trifluoromethyl)phenyl)pyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(4-(trifluoromethyl) phenyl)propanoate 0.260 g catalyzed by triethylamine dilute solution to give the intermediate 3-(2,4-difluorophenyl)-2-methyl-5-(4-(trifluoromethyl)phenyl)pyrazolo[1,5-α]pyrimidin-7-ol; the intermediate was further reacted with sodium hydroxide to obtain the title compound 22.

IR (KBr.cm−1): 3659-2840, 1612, 1565, 1479, 1326, 1173, 1104, 1063, 1014, 964, 932, 851, 791

¹H NMR (DMSO-d₆) δ7.75 (d, J=8.0, 1H), 7.72 (d, J=7.5, 2H), 7.68 (d, J=7.5, 2H), 7.07 (d, J=8.0, 1H), 6.82 (s, 1H), 6.74 (s, 1H), 2.06 (s, 3H)

### Example 23. Preparation of Potassium 3-(2,4-difluorophenyl)-2-methyl-5-(4-nitrophenyl)pyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(4-nitrophenyl) propanoate 0.237 g catalyzed by triethylamine dilute solution to give the intermediate 3-(2,4-difluorophenyl)-2-methyl-5-(4-nitrophenyl)pyrazolo[1,5-α]pyrimidin-7-ol; the intermediate was further reacted with sodium hydroxide to obtain the title compound 23.

IR (KBr.cm−1): 3664-2845, 1614, 1567, 1481, 1329, 1250, 1177, 1106, 1066, 1016

¹H NMR (DMSO-d₆) δ 8.32 (d, J=7.5, 2H), 8.05 (d, J=7.5, 2H), 7.75 (d, J=8.0, 1H), 7.07 (d, J=8.0, 1H), 6.82 (s, 1H), 6.74 (s, 1H), 2.06 (s, 3H)

### Example 24. Preparation of Potassium 3-(2,4-difluorophenyl)-5-(4-(dimethylamino) phenyl)-2-methylpyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(4-dimethylamino) propanoate 0.235 g catalyzed by dilute pyridine solution to give the intermediate 3-(2,4-difluorophenyl)-5-(4-(dimethylamino)phenyl)-2-methylpyrazolo[1,5-α]pyrimidin-7-ol; the intermediate was further reacted with sodium hydroxide to obtain the title compound 24.

IR (KBr.cm−1): 3656-2837, 1610, 1562, 1476, 1340, 1170, 1100, 1060, 1011

¹H NMR (DMSO-d₆) δ 7.75 (d, J=8.0, 1H), 7.61 (d, J=7.5, 2H), 7.07 (d, J=8.0, 1H), 6.85 (d, J=7.5, 2H), 6.82 (s, 1H), 6.74 (s, 1H), 3.06 (s, 6H), 2.06 (s, 3H)

### Example 25. Preparation of Potassium 3-(2,4-difluorophenyl)-2-methyl-5-(4-sulfamoylphenyl)pyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(4-sulfamoylphenyl) propanoate 0.271 g catalyzed by dilute pyridine solution to give the intermediate 3-(2,4-difluorophenyl)-2-methyl-5-(4-sulfamoylphenyl)pyrazolo[1,5-α]pyrimidin-7-ol; the intermediate was further reacted with sodium hydroxide to obtain the title compound 25.

IR (KBr.cm−1): 3665-2840, 1612, 1565, 1479, 1320, 1173, 1104, 1063, 1017

¹H NMR (DMSO-d₆) δ 8.07 (d, J=7.5, 2H), 7.92 (d, J=7.5, 2H), 7.75 (d, J=8.0, 1H), 7.07 (d, J=8.0, 1H), 6.82 (s, 1H), 6.74 (s, 1H), 2.06 (s, 3H), 2.0 (b, 2H)

### Example 26. Preparation of Potassium 3-(2,4-difluorophenyl)-2-methyl-5-(6-(trifluoromethyl)pyridin-3-yl)pyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(6-(trifluoromethyl) pyridin-3-yl)propanoate 0.261 g catalyzed by dilute pyridine solution to give the intermediate 3-(2,4-difluorophenyl)-2-methyl-5-(6-(trifluoromethyl)pyridin-3-yl)pyrazolo[1,5-α]pyrimidin-7-ol; the intermediate was further reacted with sodium hydroxide to obtain the title compound 26.

IR (KBr.cm−1): 3659-2840, 1612, 1565, 1479, 1326, 1173, 1104, 1063, 1018

¹H NMR (DMSO-d₆) δ 8.58 (s, 1H), 7.88 (d, J=7.5, 1H), 7.75 (d, J=8.0, 1H), 7.35 (d, J=7.5, 1H), 7.07 (d, J=8.0, 1H), 6.82 (s, 1H), 6.74 (s, 1H), 2.06 (s, 3H)

### Example 27. Preparation of Potassium 5-(6-cyanopyridin-3-yl)-3-(2,4-difluorophenyl)-2-methylpyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(6-cyanopyridin-3-yl)propanoate 0.218 g catalyzed by N-methylpiperazinedilute solution to give the intermediate 5-(6-cyanopyridin-3-yl)-3-(2,4-difluorophenyl)-2-methylpyrazolo[1,5-α]pyrimidin-7-ol; the intermediate was further reacted with sodium hydroxide to obtain the title compound 27.

IR (KBr.cm−1): 3651-2831, 2240, 1612, 1565, 1479, 1326, 1173, 1104, 1063, 1017

¹H NMR (DMSO-d₆) δ 9.20 (s, 1H), 8.35 (d, J=7.5, 1H), 7.75 (d, J=8.0, 1H), 7.73 (d, J=7.5, 1H), 7.07 (d, J=8.0, 1H), 6.82 (s, 1H), 6.74 (s, 1H), 2.06 (s, 3H)

### Example 28. Preparation of Disodium 5-(3-(2,4-difluorophenyl)-2-methyl-7-hydroxypyrazolo[1,5-α]pyrimidin-5-yl)pyridine-2-sulfinate

A procedure similar to Example 1 was employed, but the starting reactant was 4-(2,4-difluorophenyl)-3-methyl-1H-pyrazole-5-amine 0.209 g and 5-(3-ethoxy-3-oxopropionyl)pyridine-2-sulfinic acid 0.257 g catalyzed by dilute pyridine solution to give the intermediate 5-(3-(2,4-difluorophenyl)-2-methyl-7-hydroxypyrazolo[1,5-α]pyrimidin-5-yl)pyridinol-2-sulfonic acid; the intermediate was further reacted with sodium hydroxide to obtain the title compound 28.

IR (KBr.cm−1): 3666-2840, 1612, 1565, 1479, 1326, 1253, 1173, 1104, 1063, 1016

1H NMR (DMSO-d6) δ 8.19 (d, J=7.5, 1H), 7.83 (d, J=7.5, 1H), 7.75 (d, J=8.0, 1H), 7.07 (d, J=8.0, 1H), 6.88 (s, 1H), 6.74 (s, 1H), 2.06 (s, 3H), 2.0 (b, 1H)

### Example 29. Preparation of Sodium 3-(2,4-difluorophenyl)-2-methyl-5-(6-sulfamoylpyridin-3-yl)pyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(6-sulfamoylpyridin-3-yl)propanoate 0.272 g catalyzed by piperidine dilute solution to give the intermediate 3-(2,4-difluorophenyl)-2-methyl-5-(6-sulfamoylpyridin-3-yl)pyrazolo[1,5-α]pyrimidin-7-ol; the intermediate was further reacted with sodium hydroxide to obtain the title compound 29.

IR (KBr.cm−1): 3651-2840, 1612, 1565, 1479, 1330, 1173, 1063, 1021

¹H NMR (DMSO-d₆) δ 9.24 (s, 1H), 8.42 (d, J=7.5, 1H), 7.75 (d, J=8.0, 1H), 7.57 (d, J=7.5, 1H), 7.07 (d, J=8.0, 1H), 6.88 (s, 1H), 6.74 (s, 1H), 2.06 (s, 3H), 2.0 (b, 2H)

### Example 30. Preparation of Sodium 3-(2,4-difluorophenyl)-2-methyl-5-(6-phenoxypyridin-3-yl)pyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(6-phenoxypyridin-3-yl)propanoate 0.272 g catalyzed by dilute sulfuric acid to give the intermediate 3-(2,4-difluorophenyl)-2-methyl-5-(6-phenoxypyridin-3-yl)pyrazolo[1,5-α]pyrimidin-7-ol; the intermediate was further reacted with sodium hydroxide to obtain the title compound 30.

IR (KBr.cm−1): 3639-2860, 1612, 1565, 1479, 1326, 1255, 1173, 1104, 1063, 1009

1H NMR (DMSO-d6) δ 7.96-6.9 (m, 10H), 6.88 (s, 1H), 6.74 (s, 1H), 2.06 (s, 3H)

### Example 31. Preparation of Sodium 3-(2,4-difluorophenyl)-5-(4-((2-(dimethylamino)ethyl)carbamoyl)phenyl)-2-methylpyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(4-((2-(dimethylamino)ethyl)carbamoyl)phenyl)propanoate 0.272 g catalyzed by dilute sulfuric acid to give the intermediate 3-(2,4-difluorophenyl)-5-(4-((2-(dimethylamino)ethyl)carbamoyl) phenyl)-2-methylpyrazolo[1,5-α]pyrimidin-7-ol; the intermediate was further reacted with sodium hydroxide to obtain the title compound 31.

IR (KBr.cm−1): 3656-2837, 1680, 1640, 1562, 1476, 1410, 1340, 1170, 1100, 1060, 1011

¹H NMR (DMSO-d₆) δ 8.09 (d, J=7.5, 3H) 7.97 (d, J=7.5, 2H), 7.75 (d, J=8.0, 1H), 7.07 (d, J=8.0, 1H), 6.82 (s, 1H), 6.74 (s, 1H), 3.60 (t, J=7.5, 2H), 2.57 (t, J=7.5, 2H), 2.26 (s, 6H), 2.06 (s, 3H).

### Example 32. Preparation of Sodium 5-(6-Formylpyridin-3-yl)-3-(2,4-difluorophenyl)-2-methylpyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(6-Phenoxypyridin-3-yl)propanoate 0.236 g catalyzed by triethylamine dilute solution to give the intermediate 5-(6-Formylpyridin-3-yl)-3-(2,4-difluorophenyl)-2-methylpyrazolo[1,5-α]pyrimidin-7-ol; the intermediate was further reacted with sodium hydroxide to obtain the title compound 32.

IR (KBr.cm−1): 3659-2840, 1720, 1565, 1479, 1326, 1173, 1104, 1063, 1021

¹H NMR (DMSO-d₆) δ9.11 (s, 1H), 8.44 (d, J=7.5, 1H), 8.41 (d, J=7.5, 1H), 8.37 (b, 2H), 7.75 (d, J=8.0, 1H), 7.07 (d, J=8.0, 1H), 6.88 (s, 1H), 6.74 (s, 1H), 2.06 (s, 3H)

### Example 33. Preparation of Sodium 5-(6-acetoxypyridin-3-yl)-3-(2,4-difluorophenyl)-2-methylpyrazolo[1,5-α]pyrimidin-7-olate

A procedure similar to Example 1 was employed, but the starting reactant was 4-(2,4-difluorophenyl)-3-methyl-1H-pyrazole-5-amine 0.209 g and ethyl 3-oxo-3-(6-acetoxypyridin-3-yl)propanoate 0.251 g catalyzed by triethylamine dilute solution to give the intermediate 5-(6-acetoxypyridin-3-yl)-3-(2,4-difluorophenyl)-2-methylpyrazolo[1,5-α]pyrimidin-7-ol; the intermediate was further reacted with sodium hydroxide to obtain the title compound 33.

IR (KBr.cm−1): 3622-2830, 1730, 1565, 1479, 1326, 1173, 1104, 1065

¹H NMR (DMSO-d₆) δ7.97 (d, J=7.5, 1H), 7.91 (s, 1H), 7.75 (d, J=8.0, 1H), 7.07 (d, J=8.0, 1H), 6.88 (s, 1H), 6.74 (s, 1H), 6.60 (d, J=7.5, 1H), 2.28 (s, 3H), 2.06 (s, 3H)

### Example 34. Preparation of Calcium 5-(4-Cyanophenyl)-3-(2,4-difluorophenyl)-2-methylpyrazolo [1,5-α] pyrimidine-7-olate

A procedure similar to Example 1 was employed, but the starting reactant was 4-(2,4-difluorophenyl)-3-methyl-1H-pyrazole-5-amine 0.209 g and ethyl 3-(4-Cyanophenyl)-3-oxopropanoate 0.217 g catalyzed by dilute hydrochloric acid to give the intermediate 5-(4-Cyanophenyl)-3-(2,4-difluorophenyl)-2-methylpyrazolo [1,5-α] pyrimidine-7-ol; the intermediate was further reacted with sodium hydroxide to obtain the title compound 34.

IR (KBr.cm⁻¹): 3671-2840, 2220, 1610, 1566, 1479, 1327, 1175, 1106, 1065, 964

¹H NMR (DMSO-d₆) δ 7.97 (d, J=7.5, 2H), 7.82 (d, J=7.5, 2H), 7.73 (d, J=8.0, 1H), 7.07 (d, J=8.0, 1H), 6.82 (s, 1H), 6.74 (s, 1H), 2.06 (s, 3H)

### Example 35. Preparation of Calcium 3-(2,4-Difluorophenyl)-2-methyl-5-(6-(trifluoromethyl) pyridin-3-yl) pyrazolo [1,5-α] pyrimidine-7-olate

A procedure similar to Example 1 was employed, but the starting reactant was 4-(2,4-difluorophenyl)-3-methyl-1H-pyrazole-5-amine 0.209 g and ethyl 3-oxo-3-(6-(trifluoromethyl) pyridin-3-yl)propanoate 0.256 g catalyzed by dilute acetic acid to give the intermediate 3-(2,4-Difluorophenyl)-2-methyl-5-(6-(trifluoromethyl) pyridin-3-yl) pyrazolo [1,5-α] pyrimidine-7-ol; the intermediate was further reacted with sodium hydroxide to obtain the title compound 35.

IR (KBr.cm⁻¹): 3656-2840, 1612, 1565, 1479, 1326, 1173, 1104, 1063, 1018

¹H NMR (DMSO-d₆) δ 8.58 (s, 1H), 7.88 (d, J=7.5, 1H), 7.75 (d, J=8.0, 1H), 7.33 (d, J=7.5, 1H), 7.07 (d, J=8.0, 1H), 6.82 (s, 1H), 6.74 (s, 1H), 2.06 (s, 3H)

The compounds prepared in the above Examples 1-35 are shown in Table 1.

TABLE 1
Example 1-35 Preparation of Compounds and Their Molecular Weight

| Example | Chemical Structure | Molecular formula | M⁺/e |
|---|---|---|---|
| 1 | 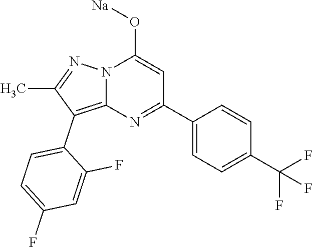 | C₂₀H₁₁F₅N₃NaO | 427.07 |
| 2 | 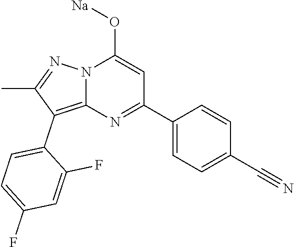 | C₂₀H₁₁F₂N₄NaO | 384.08 |
| 3 | 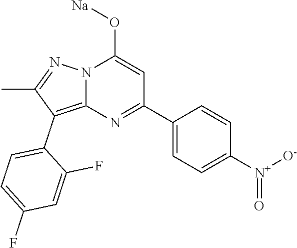 | C₁₉H₁₁F₂N₄NaO₃ | 404.07 |
| 4 | 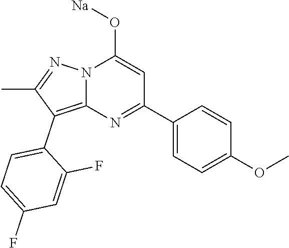 | C₂₀H₁₄F₂N₃NaO₂ | 389.10 |
| 5 | 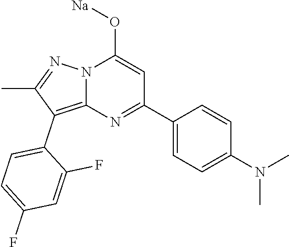 | C₂₁H₁₇F₂N₄NaO | 402.13 |
| 6 | 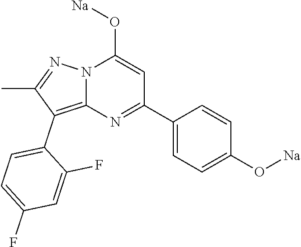 | C₁₉H₁₁F₂N₃Na₂O₂ | 397.06 |
| 7 | 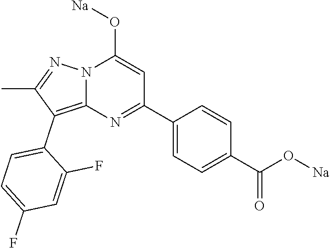 | C₂₀H₁₁F₂N₃Na₂O₃ | 425.06 |
| 8 | 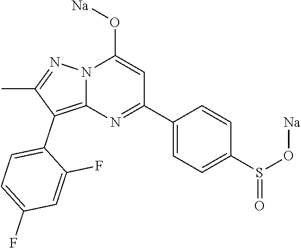 | C₁₉H₁₁F₂N₃Na₂O₃S | 445.03 |
| 9 | 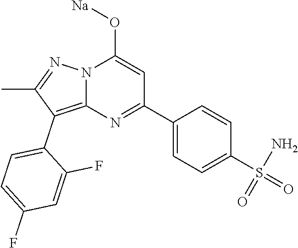 | C₁₉H₁₃F₂N₄NaO₃S | 438.06 |
| 10 | 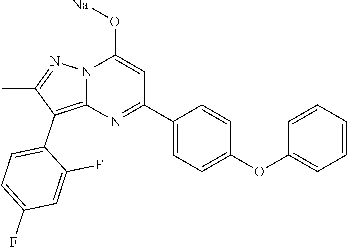 | C₂₅H₁₆F₂N₃NaO₂ | 451.11 |
| 11 | 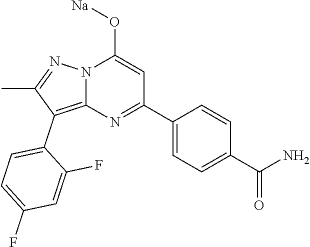 | C₂₀H₁₃F₂N₄NaO₂ | 402.09 |
| 12 | 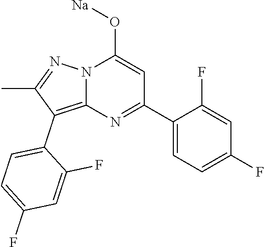 | C₁₉H₁₀F₄N₃NaO | 395.07 |
| 13 | 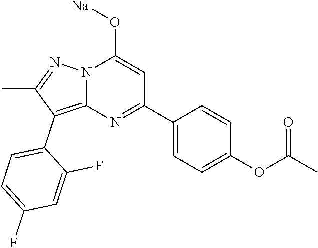 | C₂₁H₁₄F₂N₃NaO₃ | 417.09 |
| 14 | 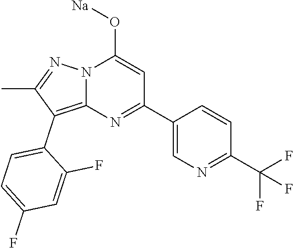 | C₁₉H₁₀F₅N₄NaO | 428.07 |
| 15 | 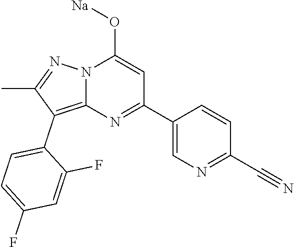 | C₁₉H₁₀F₂N₅NaO | 385.08 |
| 16 | 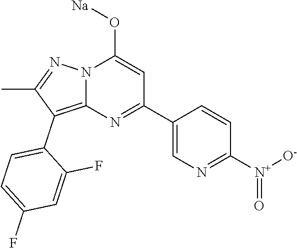 | C₁₈H₁₀F₂N₅NaO₃ | 405.06 |
| 17 | 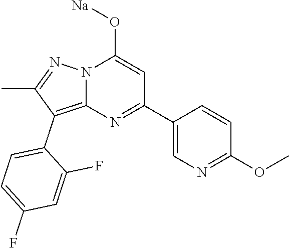 | C₁₉H₁₃F₂N₄NaO₂ | 390.09 |
| 18 | 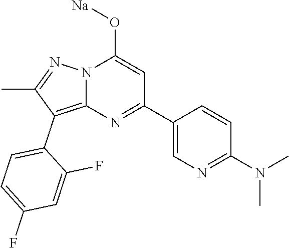 | C₂₀H₁₆F₂N₅NaO | 403.12 |
| 19 | 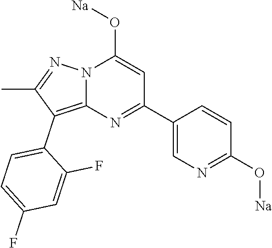 | C₁₈H₁₀F₂N₄Na₂O₂ | 398.06 |
| 20 | 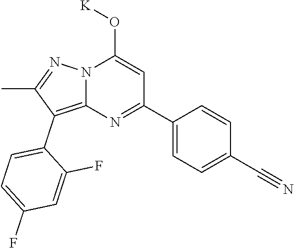 | C₂₀H₁₁F₂KN₄O | 400.05 |
| 21 | 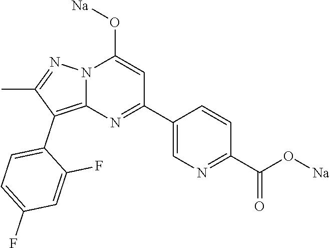 | C₁₉H₁₀F₂N₄Na₂O₃ | 426.05 |
| 22 | 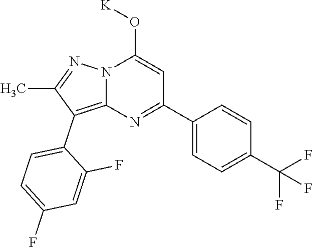 | C₂₀H₁₁F₅KN₃O | 443.05 |
| 23 | 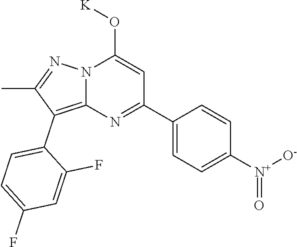 | C₁₉H₁₁F₂KN₄O₃ | 420.04 |
| 24 | 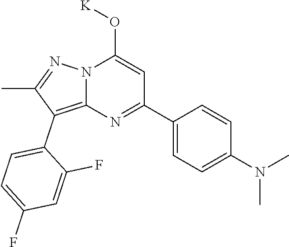 | C₂₁H₁₇F₂KN₄O | 418.10 |
| 25 | 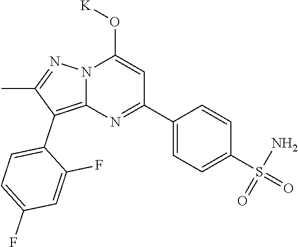 | C₁₉H₁₃F₂KN₄O₃S | 454.03 |
| 26 | 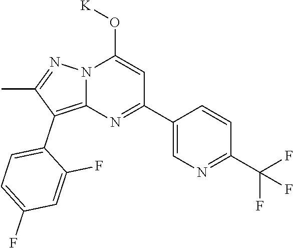 | C₁₉H₁₀F₅KN₄O | 444.04 |
| 27 | 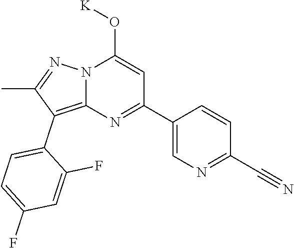 | C₁₉H₁₀F₂Ca_{1/2}N₅O | 401.05 |
| 28 | 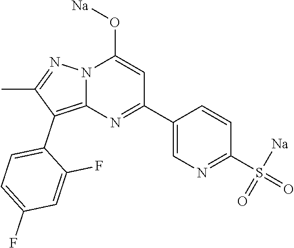 | C₁₈H₁₀F₂N₄Na₂O₃S | 446.02 |
| 29 | 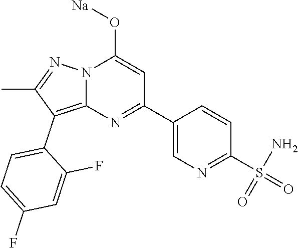 | C₁₈H₁₂F₂N₅NaO₃S | 439.05 |
| 30 | 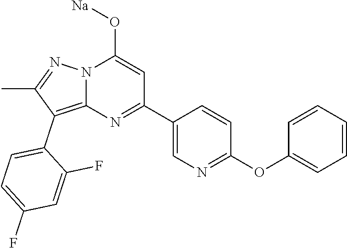 | C₂₄H₁₅F₂N₄NaO₂ | 452.11 |
| 31 | 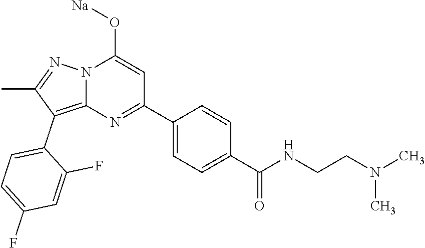 | C₂₄H₂₂F₂N₅NaO₂ | 473.16 |
| 32 | 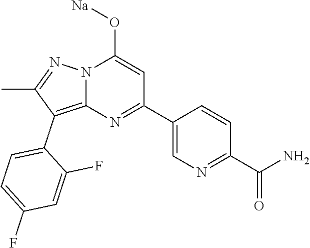 | C₁₉H₁₂F₂N₅NaO₂ | 403.09 |
| 33 | 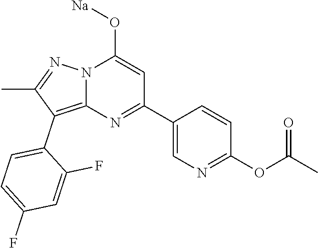 | C₂₀H₁₃F₂N₄NaO₃ | 418.09 |
| 34 | 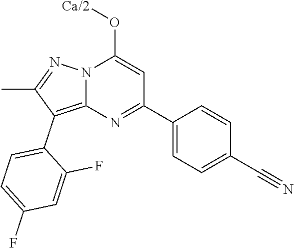 | C₂₀H₁₁F₂N₄Ca_{1\2}O | 381.32 |
| 35 | 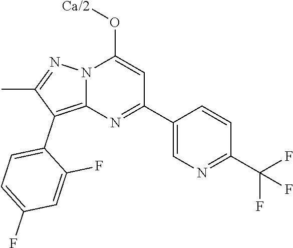 | C₁₉H₁₀F₅N₄Ca_{1\2}O | 425.30 |

### Antibacterial Studies of the Compounds In Vitro

### The Preparation of Injections

Compound 1, 5.0 g, ethanol 60 ml, 1,2-propanediol 60 ml and Tween (80) 10 ml were dissolved and the injection water was added up to total volume of 500 ml. The solution was filtered with 0.22 μm membrane filter and sterilized for 30 min at 100° C. to obtain 100 preparation of injection 5 mg/5 ml.

### Materials and Methods

1. Standard strains: Bacillus cereus 2, Bacillus subtilis 168, Enterococcus faecalis 29212, Enterococcus faecium F2518 (vre), Staphylococcus aureus 29231, Staphylococcus aureus 43300 (MRSA), Staphylococcus aureus 703 (MRSA), Streptococcus pneumoniae 6303 (PRSP), Streptococcus pneumoniae M2, Streptococcus agalactia B group, Streptococcus pneumoniae 10351, Bacillus anthracis Bacillus, Diphtheria bacilli, Clostridium perfringens, Oral Candida, Peel fungus.

2. TEST SAMPLES: Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 22, Compound 23, Compound 24, Compound 25, Compound 26, Compound 27, Compound 28, Compound 29, Compound 30, Compound 31, Compound 32, Compound 33

3. Method

(1) Sterilization: The required experimental equipment and culture solution were autoclaved at 121° C. for 30 min; sterile ultraviolet irradiation for 30 min.

(2) Bacterial Enrichment

Preparation of broth medium: Accurately weigh 6 g of tryptic soy broth medium in a 500 ml beaker, add 200 ml of distilled water, dissolve well by heating, transfer into a conical flask, add a cotton plug, and pack with autoclave.

Preparation of slant culture medium: 3.8 g of tryptic soy agar was weighed into a 500 ml beaker, 100 ml of distilled water was added, dissolved sufficiently by heating, transferred into an Erlenmeyer flask, tampon added, and autoclaved by dressing. After a little cooling, it is divided into 7 tubes, about 10-15 ml per tube, tilted at an appropriate angle, and cooled for later use.

Bacterial amplification: Open the ATCC 4300 sealed vial, take a small amount of bacterial powder block with a small ophthalmology clip after disinfection, transfer it into a 5 ml centrifuge tube, add 0.6 ml of tryptic soy broth medium, and mix well. The solution was divided into 7 slant mediums, 80 μl/test tube, and evenly coated. It was placed in an incubator and cultured at 37° C. for 24 hours.

Preparation of bacterial suspension and bacterial count: The bacteria in the large test tube were washed with the culture solution and transferred into a sterile centrifuge tube. Mix well and prepare a bacterial suspension. A drop of the pipette was taken for bacterial density determination. Take a clean blood cell counting plate, cover a counting piece on the counting area, dilute the bacterial suspension with physiological saline for a certain multiple, and blow evenly. Pipette a little, from the sides of the intermediate plate of the counting plate. A drop (not too much) is dropped into the groove along the lower edge of the cover glass, so that the bacterial suspension fills the counting area with the surface tension of the liquid, so that bubbles are not generated. The plate was placed under a microscope for bacterial count. Count the total number of bacteria in 16 cells, and calculate the concentration of the bacteria using the following formula: Bacterial density (1/ml)=16 small cells number of bacteria×10⁴×dilution factor

(3) 96-Well Plate Inoculation

Preparation of the sample: First, the drug to be tested is fully dissolved in a small amount of DMSO, and then the culture medium is formulated into a desired initial concentration, and sequentially diluted to each test gradient.

Preparation of bacterial solution: According to the measurement result of the bacterial concentration, the bacterial suspension was diluted with a culture solution (TSB) to a bacterial solution having a concentration of 1.07×10 7 cfμ/ml.

Dosing regimen: The experiment was divided into positive control group, normal saline group, blank control group and each test group. Among them, there were 6 gradient wells in each test group, saline group and blank group. The positive control group was 7 gradient holes. 50 μl of the bacterial suspension, 30 μl of the culture solution, and 20 μl of each sample solution were sequentially added to each well.

Culture and observation: The 96-well plate was added and placed in an incubator for cultivation. The culture temperature was 37° C. and the culture time was 24 hours. The cultivation was completed in a clean bench to observe the growth of colonies in each group. The bacterial liquid is clear, no turbidity, and the bottom of the well is aseptic. The concentration is determined as the minimum inhibitory concentration (MIC) of the drug.

4. Results and Conclusions

Dilute the solution (1st to 10th holes) by microdilution method in a microporous dilution plate of a 96-well plate, and then add 50 μL of the bacterial solution to the 1st to 11th holes to make the liquid concentration was reduced in multiples in the 1st to 10th holes and the 12th well was added with 100 μL of the medium as a blank control. After the microporous dilution plates were shaken and mixed, the cells were incubated at 37° C. for 24 hours in a covered porcelain dish with wet gauze, and the results were observed under a light source with a black background. The bacteria grow in a spherical shape, diffuse turbidity or a button-like precipitate at the bottom. The minimum drug concentration contained in the sterile growth well is the minimum inhibitory concentration. The results are shown in Table 2.

TABLE 2
MIC values of in vitro antibacterial test results (μM)

| Micro- | Compound | Compound | Compound | Compound | Compound | Compound | Compound | Compound | Compound |
|---|---|---|---|---|---|---|---|---|---|
| organism | 1 | 2 | 3 | 4 | 5 | 6 | 7 | 8 | 9 |
| 1 | ++ | ++ | ++ | + | + | ++ | + | + | + |
| 2 | ++ | + | ++ | − | − | ++ | − | − | ++ |
| 3 | ++ | ++ | ++ | + | + | ++ | + | + | ++ |
| 4* | + | + | + | + | + | + | + | + | + |
| 5 | ++ | ++ | ++ | − | − | ++ | − | − | ++ |
| 6* | ++ | +++ | +++ | ++ | + | ++ | ++ | + | ++ |
| 7* | + | +++ | +++ | + | + | ++ | + | + | ++ |
| 8* | ++ | + | ++ | − | − | + | − | − | + |
| 9 | ++ | + | ++ | + | ++ | + | ++ | + | ++ |
| 10 | + | + | − | − | − | + | − | − | + |
| 11 | ++ | ++ | ++ | − | − | ++ | − | − | ++ |
| 12 | − | − | − | − | − | − | − | − | − |
| 13 | − | − | − | − | − | − | − | − | − |
| 14 | − | − | − | − | − | − | − | − | − |
| 15 | ++ | + | − | + | ++ | ++ | − | + | ++ |
| 16 | − | + | − | + | + | + | + | + | − |

MIC values of in vitro antibacterial test results (μM)

| Micro- | Compound | Compound | Compound | Compound | Compound | Compound | Compound | Compound |
|---|---|---|---|---|---|---|---|---|
| organism | 10 | 11 | 12 | 13 | 14 | 15 | 16 | 17 |
| 1 | ++ | + | ++ | + | ++ | ++ | ++ | + |
| 2 | ++ | − | − | − | ++ | + | + | − |
| 3 | ++ | + | + | + | ++ | ++ | ++ | + |
| 4* | + | + | + | + | + | + | + | + |
| 5 | ++ | − | − | − | ++ | ++ | ++ | − |
| 6* | + | ++ | + | ++ | + | + | + | ++ |
| 7* | ++ | + | + | + | ++ | ++ | ++ | + |
| 8* | + | − | − | − | ++ | + | ++ | − |
| 9 | + | ++ | + | ++ | + | ++ | + | ++ |
| 10 | + | − | − | − | − | − | − | − |
| 11 | ++ | − | − | − | ++ | ++ | ++ | − |
| 12 | − | − | − | − | − | − | − | − |
| 13 | − | − | − | − | − | − | − | − |
| 14 | − | − | − | − | − | − | − | − |
| 15 | + | + | + | ++ | ++ | + | ++ | ++ |
| 16 | + | + | + | + | − | − | − | + |

MIC values of in vitro antimicrobial t results (μM)

| Micro- | Compound | Compound | Compound | Compound | Compound | Compound | Compound | Compound | Compound |
|---|---|---|---|---|---|---|---|---|---|
| organism | 18 | 19 | 20 | 21 | 22 | 23 | 24 | 25 | 26 |
| 1 | ++ | ++ | ++ | + | ++ | ++ | ++ | + | ++ |
| 2 | − | − | ++ | − | ++ | − | ++ | − | ++ |
| 3 | + | + | ++ | + | + | + | ++ | + | ++ |
| 4* | + | + | + | + | + | + | + | + | + |
| 5 | − | − | ++ | − | ++ | − | ++ | − | ++ |
| 6* | ++ | ++ | + | ++ | ++ | ++ | + | + | ++ |
| 7* | + | + | ++ | + | ++ | + | ++ | + | ++ |
| 8* | − | − | + | − | + | − | + | − | + |
| 9 | ++ | ++ | ++ | ++ | ++ | ++ | ++ | ++ | + |
| 10 | − | − | − | − | + | − | − | − | + |
| 11 | − | − | ++ | − | + | − | ++ | − | + |
| 12 | − | − | − | − | − | − | − | − | − |
| 13 | − | − | + | − | − | − | − | − | − |
| 14 | − | − | + | − | − | − | + | − | − |
| 15 | − | − | + | ++ | ++ | ++ | ++ | ++ | ++ |
| 16 | + | + | − | + | + | + | + | + | + |

MIC values of in vitro antimicrobial t results (μM)

| Micro- | Compound | Compound | Compound | Compound | Compound | Compound | Compound |
|---|---|---|---|---|---|---|---|
| organism | 27 | 28 | 29 | 30 | 31 | 32 | 33 |
| 1 | ++ | ++ | + | + | ++ | ++ | ++ |
| 2 | ++ | − | − | − | ++ | − | − |
| 3 | ++ | + | + | + | ++ | + | ++ |
| 4* | + | + | + | + | + | + | + |
| 5 | ++ | − | − | − | ++ | − | − |
| 6* | + | + | ++ | ++ | + | ++ | ++ |
| 7* | ++ | + | + | + | ++ | + | + |
| 8* | ++ | − | − | − | ++ | + | − |
| 9 | ++ | ++ | ++ | ++ | ++ | ++ | ++ |
| 10 | − | − | − | − | − | − | − |
| 11 | ++ | − | − | − | ++ | − | − |
| 12 | − | − | − | − | − | − | − |
| 13 | + | − | − | − | − | − | − |
| 14 | + | − | − | − | − | − | − |
| 15 | ++ | ++ | ++ | ++ | ++ | ++ | + |
| 16 | − | + | + | + | − | + | + |

Note :
(1) Bacteria and fungi * is resistant bacteria
1. Bacillus cereus 2;
2. Bacillus subtilis 168;
3. Enterococcus faecalis 29212;
4*. Enterococcus faecalis F2518(VRE);
5. Staphylococcus aureus 29231;
6*. Staphylococcus aureus 43300(MRSA);
7*. Staphylococcus aureus 703(MRSA);
8*. Streptococcus pneumoniae 6303 (PRSP);
9. Streptococcus pneumoniae M2;
10. Streptococcus agalactia B group;
11. Streptococcus pneumoniae 10351;
12. Bacillus anthracis Bacillus 1;
13. Diphtheria bacilli;
14. Clostridium perfringens;
15. Oral Candida;
16. Peel fungus.
(2) MIC value (μM) measured value
− represents the MIC value (μM) > 150 μg/mL;
+ represents the MIC value (μM) < 150 μg/mL;
++ represents the MIC value (μM) < 100 μg/mL;
+++ represents the MIC value (μM) < 50 μg/mL.

### In Conclusion

(1) Table 2 shows that the compounds of the examples of the present invention have excellent antibacterial activity. The particular note is that the MIC values (μM) of Example Compounds 2 and 3 for Staphylococcus aureus resistant strain 43300 (MRSA) and Staphylococcus aureus resistant 703 (MRSA), are respectively, <50 μg/mL.

(2) Compounds 1, 2, 3, 4, 6, 7, 9, 10, 11, 12, 13, 14, 15, 16, 17, 18, 19, 20, 21, 22, 23, 24, 25, 26, 27, 29, 30, 31, 32 and 33 respectively for a variety of different resistant bacteria, such as fecal enterotrophic bacteria F2518(VRE), Staphylococcus aureus resistant strain 43300(MRSA), golden yellow grape ball Drug-resistant bacteria 703 (MRSA) and pneumococcal resistance bacteria 6303(PRSP) have inhibitory effects, and their MIC values (μM) are in the range of <100 μg/mL, respectively.

(3) Compounds 1, 2, 3, 6, 9, 10, 14, 15, 16, 20, 22, 24, 26, 27 and 31 against Bacillus cereus 2, Bacillus subtilis 168, Enterococcus faecalis 29212, Enterococcus faecalis F2518(VRE), Staphylococcus aureus 29231, Staphylococcus aureus 43300(MRSA), Staphylococcus aureus 703(MRSA), Streptococcus pneumoniae 6303(PRSP) and Streptococcus pneumoniae M2 are effective. The MIC values (μM) is in the <100 μg/mL range.

(4) Compounds 1, 5, 6, 9, 13, 14, 16, 17, 21, 22, 23, 24, 25, 26, 27, 28, 29, 30, 31, 32 and 33 are effective against oral Candida. The MIC values (μM) are in the <100 μg/mL range, respectively.

### Antibacterial Studies of the Compounds In Vivo

### 1. Materials

Test sample: compound 3, compound 5, compound 9, compound 10, compound 30, compound ingredient 1, compound ingredient 2.

Test animals: kunming healthy mice, weighing 19-21 g, male and female, are divided into groups, and other groups are used by single sex. They are provided by the animal center of the institute of materia medica, beijing academy of military medical sciences.

Strain: Staphylococcus aureus 43300 (MRSA)

### 2. Method

The mice were randomly divided into a blank control group, a positive control group, and a test drug group, with 10 rats in each group, half male and half female. according to the mouse body weight 0.2 ml/10 g of the cell suspension (MRSA 2152, concentration of 5.0×106 cfμ/ml) was administered into the abdominal cavity of each mouse. bacteria (mrsa-2152), the bacterial concentration was 5.0×10 6 cfu/ml, and after 6 hours, the second administration was performed. After 30 days of observation, the survival period of each group of animals was recorded, and the life extension rate of the positive control group and the sample group was calculated:

life extension rate %=(the number of days in the test group−the number of days in the blank group)/the number of days in the blank group×100%

### 3. Results and Conclusions

TABLE 3
The average survival days and life prolongation rate

| | | | | | | | | Compound | Compound |
|---|---|---|---|---|---|---|---|---|---|
| | | | Compd | Compd | Compd | Compd | Compd | ingredient | ingredient |
| | saline | Ciprofloxacin | 3 | 5 | 9 | 10 | 30 | 1 | 2 |
| Dosage | — | 20 | 30 | 30 | 25 | 25 | 30 | 30 | 30 |
| (mg/kg) | | | | | | | | | |
| Survival | 1.3 | 3.4 | 4.0 | 2.6 | 3.0 | 3.5 | 2.4 | 4.5 | 4.8 |
| days (days) | | | | | | | | | |
| Life | — | 161.5 | 207.7 | 100.0 | 130.77 | 169.2 | 84.62 | 246.15 | 269.23 |
| extension | | | | | | | | | |
| rate (%) | | | | | | | | | |

Note:
Compound ingredient 1 is compound 30 + ciprofloxacin, ratio 1:1
Compound ingredient 2 is compound 5 + ciprofloxacin, ratio 1:1

Tests In vivo showed that Compound 3, Compound 5, Compound 9, Compound 10, Compound 30, Compound ingredient 1 and Compound ingredient 2 had a certain inhibitory effect on drug-resistant Staphylococcus aureus 43300 (MRSA), and the life extension rate exceeded 50% when administered. The life extension rate of the compound ingredient compared with the single side was significantly improved, indicating that the compound ingredient medication can significantly enhance the life extension rate. The above samples can be used as a further in-depth study of anti-MRSA.

1. A compound represented by the general formula (I), a derivative or analog thereof: 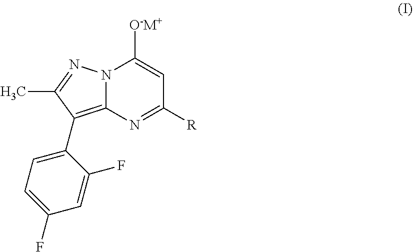
wherein M⁺ is one of Li⁺, K⁺, Na⁺, Ca^{+/2} and R is H, optionally substituted aromatic, alicyclic or fused romatic and alicyclic groups.

2. A compound according to the claim 1, wherein R is one of a group 1, 2, 3, 4, 5, 6, 7, 8 and 9; 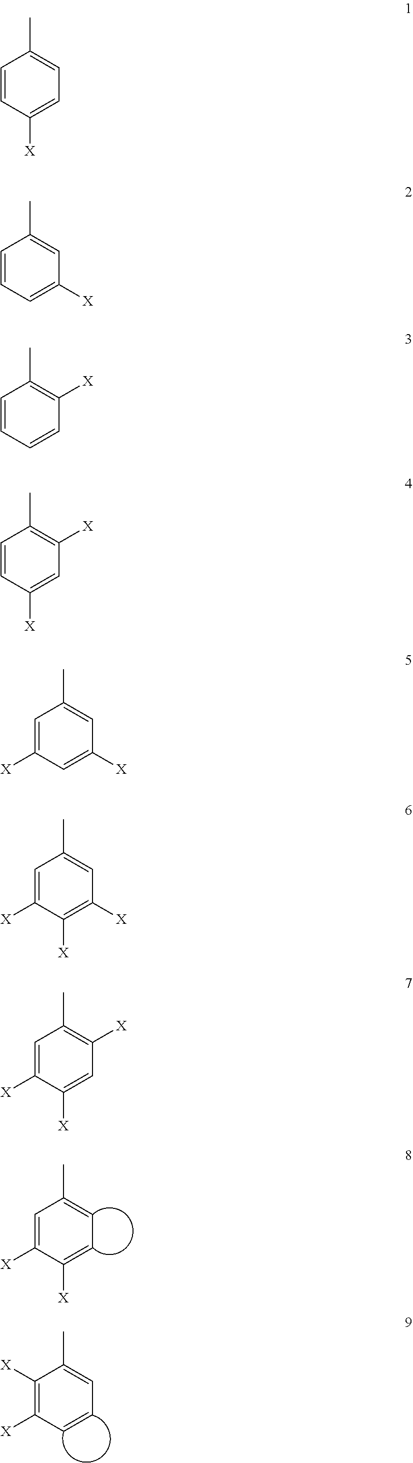
wherein the alicyclic ring fused with the aromatic ring in the chemical structure of 8 and 9 is a three-carbon, four-carbon or five-carbon alicyclic ring; X may not exist, or may be the same or different substituents: optionally substituted hydrogen, halogen, hydroxyl, mercapto, cyano, carbonyl, substituted carbonyl, aldehyde, ketone, nitro, carboxyl, substituted carboxyl, carboxylic acid ester, secondary amino, tertiary amino, two-carbon, three-carbon, four-carbon or five-carbon or six-carbon alkoxy, arylalkoxy, aryloxy, heteroaryloxy, alkylthio, mercaptoester, arylalkylthio, arylthio, heteroarylthio, ester, acyloxy, phosphoxy, sulfoxy, aryloxy, quaternary ammonium salt, amide, hydrazino, oxime, hydrazone, nitrogen-containing aliphatic, nitrogen-containing aromatic, nitrogen-containing cyclic, nitrogen-containing alicyclic, nitrogen-containing aromatic cyclic, nitrogen-containing aromatic heterocyclic, phosphide, phosphoric acid, phosphoric acid ester, one-carbon, two-carbon, three-carbon, four-carbon or five-carbon group or a combination thereof.

3. A compound according to the claim 1, wherein
a compound, a derivative or analog of the present invention selected from anyone but not limiting of the following compounds:
sodium3-(2,4-difluorophenyl)-2-methyl-5-(4-(trifluoromethyl)phenyl)pyrazolo[1,5-α]pyrimidin-7-olate, sodium5-(4-cyanophenyl)-3-(2,4-difluorophenyl)-2-methylpyrazolo[1,5-α]pyrimidin-7-olate, sodium 3-(2,4-difluorophenyl)-2-methyl-5-(4-nitrophenyl)pyrazolo[1,5-α]pyrimidin-7-olate, sodium 3-(2,4-difluorophenyl)-5-(4-methoxyphenyl)-2-methylpyrazolo[1,5-α]pyrimidin-7-olate, sodium3-(2,4-difluorophenyl)-5-(4-(dimethylamino)phenyl)-2-methylpyrazolo[1,5-α]pyrimidin-7-olate, disodium 3-(2,4-difluorophenyl)-2-methylpyrazolo[1,5-α] pyrimidine-5,7-bis(olate), disodium 4-(3-(2,4-difluorophenyl)-2-methyl-7-hydroxy-pyrazolo[1,5-α]pyrimidin-5-yl)benzoate, disodium 4-(3-(2,4-difluorophenyl)-2-methyl-7-oxidopyrazolo[1,5-α]pyrimidin-5-yl) benzenesulfonate; sodium 3-(2,4-difluorophenyl)-2-methyl-5-(4-sulfamoylphenyl)pyrazolo[1,5-α] pyrimidin-7-olate, sodium 3-(2,4-difluorophenyl)-2-methyl-5-(4-phenoxyphenyl)pyrazolo[1,5-α] pyrimidin-7-olate, sodium 5-(4-carbamoylphenyl)-3-(2,4-difluorophenyl)-2-methylpyrazolo[1,5-α] pyrimidin-7-olate, sodium 3,5-bis(2,4-difluorophenyl)-2-methylpyrazolo[1,5-α]pyrimidin-7-olate, sodium 5-(4-acetoxyphenyl)-3-(2,4-difluorophenyl)-2-methylpyrazolo[1,5-α]pyrimidin-7-olate, sodium 3-(2,4-difluorophenyl)-2-methyl-5-(6-(trifluoromethyl)pyridin-3-yl)pyrazolo[1,5-α] pyrimidin-7-olate, sodium 5-(6-cyanopyridin-3-yl)-3-(2,4-difluorophenyl)-2-methylpyrazolo[1,5-α]pyrimidin-7-olate, sodium 3-(2,4-difluorophenyl)-2-methyl-5-(6-nitropyridin-3-yl)pyrazolo[1,5-α]pyrimidin-7-olate, sodium 3-(2,4-difluorophenyl)-5-(6-methoxypyridin-3-yl)-2-methylpyrazolo[1,5-α]pyrimidin-7-olate, sodium 3-(2,4-difluorophenyl)-5-(6-(dimethylamino) pyridin-3-yl)-2-methylpyrazolo[1,5-α]pyrimidin-7-olate, disodium 3-(2,4-difluorophenyl)-2-methy 1-5-(6-hydroxypyridin-3-yl)pyrazolo[1,5-α]pyrimidin-7-olate, potassium 5-(4-cyanophenyl)-3-(2,4-difluorophenyl)-2-methylpyrazolo[1,5-α]pyrimidin-7-olate, disodium 5-(3-(2,4-difluorophenyl)-2-methyl-7-hydroxypyrazolo[1,5-α]pyrimidin-5-yl)picolinate, potassium 3-(2,4-difluorophenyl)-2-methyl-5-(4-(trifluoromethyl)phenyl)pyrazolo[1,5-α]pyrimidin-7-olate, potassium 3-(2,4-difluorophenyl)-2-methyl-5-(4-nitrophenyl)pyrazolo[1,5-α]pyrimidin-7-olate, potassium 3-(2,4-difluorophenyl)-5-(4-(dimethylamino)phenyl)-2-methylpyrazolo[1,5-α]pyrimidin-7-olate, potassium 3-(2,4-difluorophenyl)-2-methyl-5-(4-sulfamoylphenyl)pyrazolo[1,5-α]pyrimidin-7-olate, potassium 3-(2,4-difluorophenyl)-2-methyl-5-(6-(trifluoromethyl)pyridine-3-yl)pyrazolo[1,5-α]pyrimidin-7-olate, potassium 5-(6-cyanopyridin-3-yl)-3-(2,4-difluorophenyl)-2-methylpyrazolo[1,5-α]pyrimidin-7-olate, disodium 5-(3-(2,4-difluorophenyl)-2-methyl-7-hydroxypyrazolo[1,5-α]pyrimidin-5-yl)pyridine-2-sulfinate, sodium 3-(2,4-difluorophenyl)-2-methyl-5-(6-sulfamoylpyridin-3-yl)pyrazolo[1,5-α]pyrimidin-7-olate, sodium 3-(2,4-difluorophenyl)-2-methyl-5-(6-phenoxypyridin-3-yl)pyrazolo[1,5-α]pyrimidin-7-olate, sodium 3-(2,4-difluorophenyl)-5-(4-((2-(dimethylamino)ethyl)carbamoyl)phenyl)-2-methylpyrazolo[1,5-α] pyrimidin-7-olate, sodium 5-(6-carbamoylpyridin-3-yl)-3-(2,4-difluorophenyl)-2-methylpyrazolo[1,5-α] pyrimidin-7-olate, sodium 5-(6-acetoxypyridin-3-yl)-3-(2,4-difluorophenyl)-2-methylpyrazolo[1,5-α]pyrimidin-7-olate, calcium 5-(4-Cyanophenyl)-3-(2,4-difluorophenyl)-2-methylpyrazolo[1,5-α]pyrimidine-7-olate, calcium 3-(2,4-Difluorophenyl)-2-methyl-5-(6-(trifluoromethyl) pyridin-3-yl)pyrazolo[1,5-α]pyrimidine-7-olate.

4. A compound according to the claim 1, wherein:
a process for the manufacture of a compound of formula I comprises:
i) For the preparation of the key intermediates (3) by the cyclization of the compound (1) with the compound (2) containing the R group at a temperature of −40 to 180° C. in an organic solvent under the catalysis 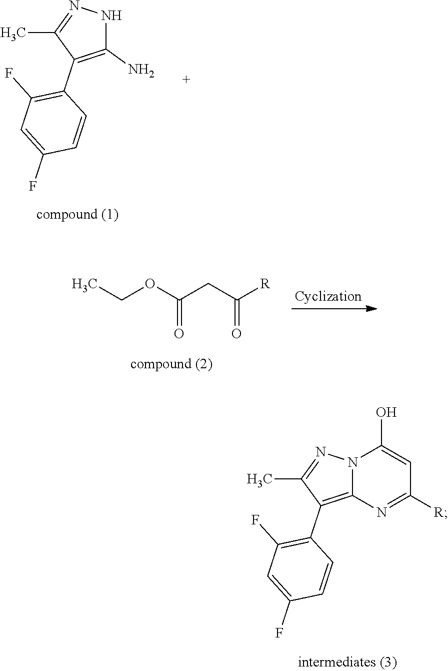
ii) For the preparation of the compound (I) by the reaction of the key intermediates (3) with an alkali or an alkali metal at a temperature of −40 to 180° C. in an organic solvent under the catalysis.

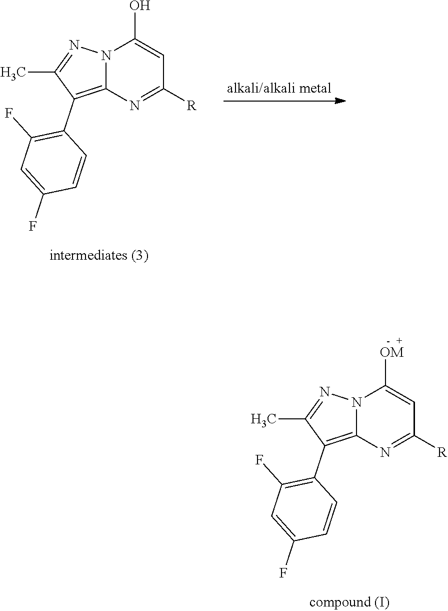

5. A compound according to the claim 4, wherein:
the process in the step i), the catalyst is an acid or a base catalyst and the acid catalyst is selected from the group consisting of hydrochloric acid, sulfuric acid, nitric acid, fumaric acid, maleic acid, succinic acid, acetic acid, citric acid, tartaric acid, carbonic acid, phosphoric acid and oxalic acid; the base is selected from the group consisting of sodium carbonate, sodium hydride, sodium hydroxide, potassium hydroxide, triethylamine, pyridine, piperidine and N-methylpyrazine; the solvent is selected from one of tetrahydrofuran, 1,4-dioxane, acetonitrile, N,N-dimethylformamide, N,N-dimethylacetamide, n-hexane and toluene.

6. The process method according to the claim 5, wherein:
the solvent is N,N-dimethylformamide.

7. The process method according to the claim 4, wherein in the step ii):
the solvent is tetrahydrofuran, 1,4-dioxane, acetonitrile, N,N-dimethylformamide, N,N-dimethylacetamide or one of the N,N-dimethylacetamide mixed with water; the base is selected from the group consisting of sodium carbonate, sodium hydride, potassium hydroxide, sodium hydroxide or calcium hydroxide, and the alkali metal is selected from the group consisting of Li, Na or K.

8. The process method according to the claim 7, wherein:
the solvent is a mixed solvent of tetrahydrofuran and water; the base is sodium hydroxide.

9. A method of treating, preventing or slowing the progression of bacteria, fungi and other infection diseases associated with a bacterial or fungal infection, comprising:
a therapeutically effective amount of the compound (I), a derivative or an analog according to the claim 1 thereof is administered to a patient in need of such treatment.

10. The method according to claim 9, wherein:
the bacterium is a Gram-positive bacteria: Staphylococcus, pneumococci, Enterococcus faecalis, Streptococcus, Streptococcus bovis, Streptococcus pneumoniae, Streptococcus pneumoniae, Streptococcus pneumoniae, Streptococcus pneumoniae, Streptococcus pyogenes, Streptococcus agalactiae, Streptococcus mutans, Streptococcus bovis, Streptococcus agalactia B, Streptomyces, diphtheria, tetanus, Escherichia, Bacillus anthracis, tetanus, Bacillus cereus, Bacillus subtilis, Clostridium, Bacillus cereus, Bacillus subtilis, Bacillus anthracis, diphtheria, Clostridium, tetanus, Clostridium perfringens, Clostridium perfringens, Atinomycetes, tuberculosis.

11. The method according to claim 10, wherein:
the bacterium is a Gram-positive resistant bacterium: methicillin-resistant Staphylococcus, vancomycin-resistant Staphylococcus aureus, Staphylococcus-induced clindamycin resistance, Vancomycin enterococcus, Enterococci high level of aminoglycoside resistance, penicillin-resistant Streptococcus pneumoniae, multi-drug resistant Acinetobacter baumannii, drug resistance and multi-drug resistant Mycobacterium tuberculosis, Mycobacterium tuberculosis, Streptococcus, Enterococcus faecalis, Pseudomonas aeruginosa, Escherichia coli, Acinetobacter baumannii, Haemophilus influenzae-resistant Haemophilus, Neisseria gonorrhoeae, Neisseria meningitidis, Enterobacteriaceae-resistant bacteria, drug-resistant Patina monocytogenes.

12. The method according to claim 9, wherein:
a compound for treating, preventing or slowing the progression of bacteria, fungi and other infection diseases are the inflammation and inflammatory diseases, viral diseases and immune system diseases accompanied by the infection of bacterial, fungal and concurrent viral due to bacterial and fungal infections.

13. The method according to claim 12, wherein:
the diseases is selected from infections of upper and lower respiratory tract, infections of skin and soft tissue, urinary tract infections, sepsis, endocarditis caused by methicillin-sensitive Staphylococcus, Hemolytic Streptococcus and Streptococcus pneumoniae; Haemophilus influenzae, Proteus mirabilis, large intestine urinary tract infections caused by bacterium-sensitive strains and pneumonia; respiratory infections, urinary tract infections, skin soft tissues caused by Streptococcus, Streptococcus pneumoniae, and susceptible strains of Haemophilus influenzae, Escherichia coli, and Proteus mirabilis infection, sepsis, bone and joint infections and abdominal and pelvic infections; infection of hemolytic streptococcus, pneumococci, sensitive Staphylococcus aureus; endocarditis caused by Streptococcus viridans and enterococcus; and gangrene, anaerobic bacterial infection, anthrax, syphilis, gonorrhea.

14. According to claims 9 and 1, wherein:
a method of treating, preventing or slowing the progression of diseases with a compound (I), a derivative or an analog is administered together with at least one known drug selected from:
β-lactams: penicillin, procaine penicillin, benzathine penicillin, methicillin, oxacillin, cloxacillin, dicloxacillin sodium, ampicillin, amoxicillin, hetacillin, carbenicillin, sulbenillin, temocillin, furazocillin, piperacillin, azlocillin, mezlocillin, ticarcillin, mecillin, apacillin, apocillin, lenampicillin, flucloxacillin, sulbaccillin, piramacillin, acesulfamecil, bacilcillin, carocycline, furbuterazine, ceftriaxone, cefpirome, cefuroxime, cefuroxime axetil, cefotaxime, cefotaxime, cefotaxime, cephalosporin thiopurine, cefacid, cefpirin, cefazolin, cefmenoxime, cefoperazone, cefaclor, ceftizoxime, ceftazidime, cefonicid, cefdinir, cefixime, cefradine, cefpi amine, cefmenoxime, ceftiram, cefpodoxime, cefdiprozil, cefotiam, cefetamet, ceftizox, cefprozil, ceftibuten, cefepime, cephalexin, cephradine, cefaclor, ceftriaxone, cephalosporin, ampicillin, cefmenudene, cefsulodin, cefoxitin, cefmetazole, cefotetan, cefminox, cephalosporin, cefaclor, cefazolin, cefotiam, ceftriax, cefixime, cefotaxime, chlorocarbon cephalosporin, fluoxetine; macrolides: dirithromycin, roxithromycin, ropoxmycin, clarithromycin, fluoroerythromycin, azithromycin, rotamycin, tacomus, erythromycin, erythromycin, clarithromycin, guitarmycin, melamycin, columnar leucomycin, medimycin, azithromycin, josamycin, spiramycin, acetylspira aminoglycosides: netilmicin, astemizine, arbekacin, isepamicin streptomycin, caramycin, gentamicin, tobramycin, amikacin, cisplatin michelin, neomycin, paromomycin, formimicin, small nomitellin, isepamicin, dibemimethine, dardimethine, spectinomycin, streptomycin, kanamycin, etimicin, dibekacin; amide alcohols: chloramphenicol, amber chloramphenicol, palm chloramphenicol, thiamphenicol, lincomycin, clindamycin, gram polyphosphates; polypeptides: cyclosporine, teicoplanin, polymycin, polymyxin, vancomycin, norvancomycin, bacitracin, polymyxin b, fusidic acid, micammycin; rifamycin: rifabutin, rifapentine, rifaximin, rifampicin, rifamycin, rifampicin; quinolones: enoxacin, toloxacin, norfloxacin, ciprofloxacin, lomefloxacin, serfloxacin, pefloxacin, fleroxacin, temafloxacin, haloxacin, moxifloxacin, valvafloxacin, gepafloxacin, ofloxacin, levofloxacin, pazufloxacin, rufloxacin, sulfisoxazole, sulfamethoxazole, sulfadiazine, sodium sulfacetamide, silver sulfadiazine, trimethoprim, pipemidic acid, nitrofurantoin, furazolidone, nalidixic acid, ofloxacin, gatifloxacin, pazufloxacin, troxafloxacin, moxifloxacin; tetracyclines: tetracycline, terpene, minocycline, chlortetracycline, doxycycline, oxytetracycline, doxycycline, metacycline, dimecycline, indomethacin, beta-lactamase inhibitor clavulanic acid, sulbactam, tazobactam; carbapenem antibiotics: imipenem, cilastatin, panipenem, betamipron, meropenem, cefomycin; sulfonamides: sulfametholone, sulfamethazine, sulfamethazine, sulfamethoxazole, sulfamonomethoxine, iodine-p-methoxypyrimidine, iodine, sulfaguanidine, zinc sulfadiazine, sulfamethamine, amber sulfonamide thiazole, thiazolidine, sulfamethoxazole, thienamycin, clavulanic acid, aztreonam, imipenem, faropenem, cilastatin, sulbactam, tazobactam, carumimo south, sirmimycin, chloramphenicol palmitate, fosfomycin, SV, bromoprolin, octenidine, urotropine, montadylamine, bismuth subsalicylate, metronidazole phosphate disodium, sulpirone, new sterilization, metronidazole, arubicin, epirubicin, zorubicin, pirarubicin, idarubicin, mupirocin, nimidazole, tinidazole, pipemidic acid, nitrofurantoin; nitrofuran: furazolidone, trimethoprim; methylfuran: sulfasalazine; antifungal: thiconazole, lanoconazole, noconazole, butoconazole, chlorconazole, fenteconazole nitrate, sheraconazole, oxyxazole, bifonazole, fluconazole, itraconazole, saconazole, clotrimazole, econazole, tioconazole, miconazole, ketoconazole, naftifine, butenafine, ciclopirox, amorolfine, amphotericin B, erythromycin, flucyto sine, terbina fen, nystatin, griseofulvin, and flunin.lactams:penicillin, procaine penicillin, benzathine penicillin, methicillin, oxacillin, cloxacillin, dicloxacillin, hetacillin, sulbenicillin, temocillin, mecillinam, piperacillin, ticarcillin, ticarcillin, flucloxacillin, sultamicillin, phenoxymethylpenicillin, bacampicillin, ticarcillin, talampicillin, furbenicillin, aspoxicillim, pivampicilli, meticillin, nafcillin, pivmecillinam, lenampicillin varacillin, apalcillin, carindacillin, carbenicillin, ceftriaxone, cefpirome, cefuroxime, ceftazidime, cefotaxime, cephalothin, cefathiamidine, cephalosporin, cephalosporin, cefazolin, cefmenoxime, cefoperazone, cefaclor, ceftizoxime, cefdinir, cefixime, cephradine, cefpiramide, cephalosporin, cephalosporin, cefpodoxime, cefodizime, cefotiam, cefetamet, cefprozil, cephalosporin, cefepime, cephalexin, cephradine, cefadroxil, cefoxitin, cefmeta-zole, cefotetan, cefminox, cephalosporin, cefozopran, cephalosporin, cefalotin, cefaloglycine, cefale-xin, cefradine, cefacetrile cefapirin, cefadril, cefroxadine, ceftezole, cefonicid, cefamandole, cefbu-perazone, cefdinir, cefzon, cefcapene, cefotaxime, cefteram, cefsulodine, latamoxef, cefpimizole, cefuzonam, aztreonam, erythromycin, roxithromycin, adriamycin, clarithromycin, fluoerythromycin, azithromycin, kitasamycin, albomycin, leucomycin, josamycin, spiramycin, acetyl spiramycin, netilmicin, isepamicin, streptomycin, gentamicin, tobramycin, amikacin, sisomicin, neomycin, neomycin, spectinomycin, streptomycin, kanamycin, chloramphenicol, thiamphenicol, lincomycin, clindamycin, clindamycin, cyclosporine, teicoplanin, vancomycin, teicoplanin, bacitracin, polymyxin B, rifamycin, rifabutin, rifapentine, rifaximin, rifampicin, rifamycinn, enoxacin, norfloxacin, ciprofloxacin, lome-floxacin, sparfloxacin, pefloxacin, fleroxacin, moxifloxacin, ofloxacin, levofloxacin, rufloxacin, isoxazole, sulfamethoxazole, sulfadiazine, trimethoprim, pipemidic acid, nitrofurantoin, furazolidone, nalidixic acid, difloxacin, gatifloxacin, pazufloxacin, moxifloxacin acid, tetracycline, minocycline, chlortetracycline, doxycycline, oxytetracycline, doxycycline, metacycline, clavulanic acid, sulbactam, tazobactam, imipenem, cilastatin, panipenem, betamipron, meropenem, cephamycin, sulfamethoxazole, sulfamethazine, sulfonamides, sulfamonomethoxine, sulfamethoxazole, sulfathiazole, sulfamethoxazole, sulfathiazole, thienamycin, aztreonam, faropenem, cilastatin, tazobactam, streptomycin, neomycin, kanamycin, neomycin, clindamycin, fosfomycin, brodimoprim, metronidazole, aclarubicin, epirubicin, pirarubicin, mupirocin, tinidazole, sulfasalazine, itraconazole, bifonazole, fluconazole, clotrimazole, econazole, miconazole, ketoconazole, naftifine, butenafine, ciclopirox, amorolfine, amphotericin b, flucytosine, terbinafine, nystatin, gentamycin, nebramycin, micronomicin, ribostamycin, astromicin, dibekacin, etimicin, nofloxacin, cotrim, sulfamethoxazole, mafenide, brodimoprim clafalix, laurylin, azitromycin, midecamycin, acetylmidecamycin, rokitamycin, meleumycin, methacycline, doxycyclme, demeclocycline, nifuratel, methylmercadone, norvancomycim, colistin, gramicidin, isoniazid, ethambutol, pyrazinamide, rifamycin, rifandin, viomycin, rifampin, capreomycin, ethionamide, terconazole, fenticonazole, sulconazole, fluorocytosine, ciclopirox, mepartricin, exalamide, terbinafine, ribavirin, acyclovir, ganciclovir, indinavir, nelfinavir, ritonavir, cidofovir, penciclovir, buciclovir, penciclovir, famciclovir, valaciclovir, famotine, vidarabine, zidovudine, azidothymidine, foscarnet, delavirdine, moroxydine, idoxuridine, amantadine, interferons, rimantadine, clindamycim.

15. According to claims 9 and 1, wherein:
a method of treating, preventing or slowing the progression of diseases with a compound (I), a derivative or an analog is administered at a dose of from 0.02 mg/kg to 250 mg/kg.

16. According to claims 9 and 1, wherein:
a method of treating, preventing or slowing the progression of diseases with a compound (I), a derivative or an analog is administered by oral, parenteral, subcutaneous, intravenous, intramuscular, intraperitoneal, transdermal, buccal Intrathecal, intracranial, intranasal or topical administration.
